SEQUENCE LISTING (32 sequences)

SEQ ID NO: 1 - DNA, 20 bp, Artificial Sequence
  Forward Primer Sequence for COL2
cgggtgaacgtggagagaca

SEQ ID NO: 2 - DNA, 20 bp, Artificial Sequence
  Reverse Primer Sequence for COL2
gtccagggttgccattggag

SEQ ID NO: 3 - DNA, 22 bp, Artificial Sequence
  Forward Primer Sequence for KRT8
accaggagctcatgaatgtcaa

SEQ ID NO: 4 - DNA, 17 bp, Artificial Sequence
  Reverse Primer Sequence for KRT8
tcgccctccagcagctt

SEQ ID NO: 5 - DNA, 19 bp, Artificial Sequence
  Forward Primer Sequence for SNAP25
ggcttcatccgcagggtaa

SEQ ID NO: 6 - DNA, 22 bp, Artificial Sequence
  Reverse Primer Sequence for SNAP25
gctccaggttttcatccatttc

SEQ ID NO: 7 - DNA, 19 bp, Artificial Sequence
  Forward Primer Sequence for CDH2
gccatcaagccagttggaa

SEQ ID NO: 8 - DNA, 20 bp, Artificial Sequence
  Reverse Primer Sequence for CDH2
tgcagatcgaaccgggtact

SEQ ID NO: 9 - DNA, 20 bp, Artificial Sequence
  Forward Primer Sequence for ACAN
ggcatcgtgttccattacag

SEQ ID NO: 10 - DNA, 20 bp, Artificial Sequence
  Reverse Primer Sequence for ACAN
actcgtccttgtctccatag

SEQ ID NO: 11 - DNA, 21 bp, Artificial Sequence
  Forward Primer Sequence for SOSTDC1
gttcaagtaggctgccgagaa

SEQ ID NO: 12 - DNA, 19 bp, Artificial Sequence
  Reverse Primer Sequence for SOSTDC1
gcactggccgtctgagatg

SEQ ID NO: 13 - DNA, 17 bp, Artificial Sequence
  Forward Primer Sequence for GAPDH
tgccgcctggagaaacc

SEQ ID NO: 14 - DNA, 18 bp, Artificial Sequence
  Reverse Primer Sequence for GAPDH
cgcctgcttcaccacctt

SEQ ID NO: 15 - DNA, 20 bp, Artificial Sequence
  Forward Primer Sequence for hGAPDH
gagtcaacggatttggtcgt

SEQ ID NO: 16 - DNA, 20 bp, Artificial Sequence
  Reverse Primer Sequence for hGAPDH
ttgattttggagggatctcg

SEQ ID NO: 17 - DNA, 20 bp, Artificial Sequence
  Forward Primer Sequence for hRUNX2
acagtagatggacctcggga

SEQ ID NO: 18 - DNA, 19 bp, Artificial Sequence
  Reverse Primer Sequence for hRUNX2
tgaggcggtcagagaacaa

SEQ ID NO: 19 - DNA, 18 bp, Artificial Sequence
  Forward Primer Sequence for hALP
cgcacggaactcctgacc

SEQ ID NO: 20 - DNA, 18 bp, Artificial Sequence
  Reverse Primer Sequence for hALP
gccaccaccaccatctcg

SEQ ID NO: 21 - DNA, 20 bp, Artificial Sequence
  Forward Primer Sequence for hBMP2
gaggtcctgagcgagttcga

SEQ ID NO: 22 - DNA, 20 bp, Artificial Sequence
  Reverse Primer Sequence for hBMP2
tctctgtttcaggccgaaca

SEQ ID NO: 23 - DNA, 20 bp, Artificial Sequence
  Forward Primer Sequence for SOX9
cgccatcttcaaggcgctgc

SEQ ID NO: 24 - DNA, 20 bp, Artificial Sequence
  Reverse Primer Sequence for SOX9
cctgggattgccccgagtgc

SEQ ID NO: 25 - DNA, 22 bp, Artificial Sequence
  Forward Primer Sequence for Col2
ggcaatagcaggttcacgtaca

SEQ ID NO: 26 - DNA, 22 bp, Artificial Sequence
  Reverse Primer Sequence for Col2
cgataacagtcttgccccactt

SEQ ID NO: 27 - DNA, 20 bp, Artificial Sequence
  Forward Primer Sequence for ACAN
acagctggggacattagtgg

SEQ ID NO: 28 - DNA, 20 bp, Artificial Sequence
  Reverse Primer Sequence for ACAN
gtggaatgcagaggtaattt

SEQ ID NO: 29 - DNA, 21 bp, Artificial Sequence
  Forward Primer Sequence for hPAX1
aagccgccctattcctacatc

SEQ ID NO: 30 - DNA, 18 bp, Artificial Sequence
  Reverse Primer Sequence for hPAX1
gcgcttggtgggtgaact

SEQ ID NO: 31 - DNA, 21 bp, Artificial Sequence
  Forward Primer Sequence for hFOXF1
aagccgccctattcctacatc

SEQ ID NO: 32 - DNA, 18 bp, Artificial Sequence
  Reverse Primer Sequence for hFOXF1
gcgcttggtgggtgaact

(19) United States
(12) Patent Application Publication
(10) Pub. No.: US 2024/0058507 A1
(43) Pub. Date: Feb. 22, 2024

(54) A BIOMATERIAL WITH A HIGH GLYCOSAMINOGLYCAN/HYDROXYPROLINE RATIO, COMPOSITION, METHODS AND APPLICATIONS

(71) Applicant: THE UNIVERSITY OF HONG KONG, Hong Kong Special Administrative Region (CN)

(72) Inventors: Pui Barbara CHAN, Hong Kong (CN); Xing Xing YANG, Hong Kong (CN)

(21) Appl. No.: 18/270,069

(22) PCT Filed: Jan. 10, 2022
(86) PCT No.: PCT/CN2022/071012

§ 371 Date: Jun. 28, 2023

Related U.S. Application Data
(60) Provisional application No. 63/135,018, filed on Jan. 8, 2021.

(51) Int. Cl. A61L 27/38; A61L 27/20; A61L 27/24; A61L 27/26

(52) U.S. Cl. CPC A61L 27/3856; A61L 27/20; A61L 27/24; A61L 27/26; A61L 27/3834; A61L 2430/06; A61L 2430/38

(57) ABSTRACT

A composition comprising a glycosaminoglycan component, and one or more extracellular matrix components forming a precipitate with the glycosaminoglycan component, wherein the precipitate has a glycosaminoglycan to hydroxyproline ratio from about 1:10 to about 100:1. In particular, it is a GAG-rich composition with controllable and high glycosaminoglycan (GAG) content that mimics the extracellular matrix (ECM) of GAG-rich native tissues. Also provided is a method of making and using the aforesaid composition. Further provided is a method of treating a tissue disorder in a subject using the aforesaid composition. The composition may be used as scaffolds for applications such as microcarriers, 3D culture substrates, swelling agent, volume filling agent, replacement of nucleus pulposus, cartilage, and other GAG-rich tissues.

This international patent application claims the benefit of U.S. Provisional Patent Application No.: 63/135,018 filed on Jan. 8, 2021, the entire content of which is incorporated by reference for all purpose.

### FIELD

The present disclosure relates generally to a series of novel biomaterials including, but not limited to, a composition comprising a glycosaminoglycan (GAG) component and an extracellular matrix (ECM) component. In particular, the invention relates to a composition having precipitate formed by the GAG component and the ECM component such as collagen, hyaluronic acid (HA), at controllable amounts and the precipitate has a GAG/HYP ratio. More specifically, it relates to composition, materials, methods of preparation and applications of the novel GAG-immobilized biomaterial.

### BACKGROUND

Proteoglycans are commonly found in ECM of GAG-rich tissues such as nucleus pulposus, cartilage, neurological tissue, synovial fluid, vitreous fluid, heart valves, lungs, liver, skin, blood vessels, and other tissues. They are formed by binding sulfated GAG to core proteins of proteoglycan molecules. GAG is a polysaccharide composed of a disaccharide chain rich of negative charge, facilitating water retention. Hence, GAG can bind enormous amounts of water, maintain hydration, and act as a space holder in GAG-rich tissues.

In native GAG-rich tissue, GAG forms larger ‘bottlebrush’ like proteoglycan aggregates with hyaluronic acid (HA), and the proteoglycan aggregates are distributed in the collagen meshwork. One critical parameter monitoring the normal function of GAG-rich tissue is the GAG/Hydroxyproline (HYP, presents collagen) ratio, which is the relative abundance of GAG-rich matrix to collagen meshwork. The GAG/HYP ratio is a good indicator of the quality of GAG-rich tissues such as interverbal disc, cartilage and other tissues. Due to the importance of GAG in tissue functions, the development of tissue engineering scaffolds with high GAG content to mimic the composition, structure, and function of native tissue will be valuable for GAG-rich tissue engineering. However, it is difficult to immobilize and maintain GAG in solid collagen meshwork in vitro as GAG is a highly hydrophilic polysaccharide chain that is highly soluble in water.

### SUMMARY

Provided herein is a composition of a series of GAG-rich biomaterial derived from ECM components, including, but not limited to, HA, collagen, and GAG. The invention relates to a controllable GAG composition, e.g. aminated collagen-aminated HA-GAG (aCol-aHA-GAG) and associated biomaterials. This type of biomaterials may be fabricated by chemically modifying collagen and HA, and reacting them with GAGS preferably anionic GAGS, giving rise to complex ECM structures with controllable and suitable GAG/HYP ratio that is exceptionally suitable for mimicking native tissue matrix or for cell culturing or the like. The precipitate particular coprecipitate may be in the form of nanosized ‘beads’ like and ‘bottlebrush’ like ultrastructure, and has good biocompatibility, and structurally and functionally mimic the native GAG-rich tissues such as young adult nucleus pulposus (NP), cartilage and other tissues.

In some embodiments, a series of novel biomaterial namely aminated collagen-aminated HA-GAG (aCol-aHA-GAG) and associated structures with extremely high and controllable GAG/HYP ratio has been developed that structurally and functionally mimic the characteristics of GAG-rich native tissues such as NP of intervertebral disc and cartilage. The GAG-rich composition is produced by amination modification of the extracellular matrix (ECM) and assembly of the aminated ECM components with anionic GAGs to form coprecipitates. The aCol-aHA-GAG can be formed with controllable GAG/HYP ratio, biomimetic composition and structural characteristics, and good biocompatibility by reacting the chemically modified aminated moieties with the negatively charged GAG moieties. The composition shows high-density GAGS, in a controlled manner with physiologically relevant biomimetic ultrastructures, good biocompatibility, biomechanical properties and functions such as reduced elastic modulus and fluid replacement function. As a result, the said composition is ideal for biomedical applications including but are not limited to 3D culture substrate, delivery device and scaffolding for biomolecular, cell and tissue engineering therapies for GAG-rich tissues such as nucleus pulposus, cartilage and other tissues.

The composition uses a formulation including one or more ECM components and chemical modification reagents. In the preferred embodiment, the ECM component, being capable of providing support to the cells and interacting with the cells, permitting cell migration and penetration, and facilitating the formation of proteoglycan complexes structure, is HA, collagen, GAG, or other material that supports cell growth and migration and support GAG linking and immobilization, such as fibronectin, laminin, a core protein, a link protein, and peptides including, but not limited to, self-assembled peptides (SAM) and synthetic peptide sequences such as functional epitopes of ECM component including but not limited to Arg-Gly-Asp(RGD) peptide, Arg-Gly-Asp-Ser (RGDS), Gly-Arg-Gly-Asp-Ser-Pro (GRGDSP) for fibronectin, Gly-Phe-Pyrrolysine-Gly-Glu-Arg (GFOGER) for collagen, and Ile-Lys-Val-Ala-Val (IKVAV) for laminin. These ECM components can be modified to control the density of surface charge by amination chemical modification. These modified aminated ECM components and GAGS can interact in such a way that the self-assembled co-precipitation leads to a change in physical properties of the biomaterial such as the volume, ultra-structure, morphology, ECM density, GAG/HYP ratio, GAG retention ability, mechanical property and stability, mimicking native GAG-rich tissues.

The composition can be fabricated by chemical modification, which includes exposing the species such as HA or collagen reacting with amination reagents in specific amination reagents, pH, molar ratio, concentration, temperature, crosslinker, the concentration of crosslinker, and reaction time. The amination reagents used are chemicals that contain amino groups such as ethylenediamine (EDA), tris(2-aminoethyl) amine (TAEA), L-arginine, metformin, and polyamines, etc. Crosslinker used include 1-ethyl-3(3-dimethylaminopropyl) carbodiimide (EDC) in DI water and EDC/N-Hydroxy succinimide (NHS) in 2-(N-morpholino) ethane sulfonic acid (IVIES) buffer solution. ECMs, amination reagents, and crosslinker are mixed thoroughly. In addition, the unreacted amination chemicals can be removed by, but not limited to, dialysis. Aminated ECMs are collected after removing the unreacted chemicals.

The composition can be fabricated through mixing the components, vortexing and centrifugation. The GAG-rich coprecipitate can be fabricated by different aminated ECMs mixture. The surface charge density of ECM components depends on the amination reagent concentration during the amination process that ECM components reacted with a higher concentration of amination reagents with higher GAGS incorporation. The size, GAGS retention property, and mechanical property of the coprecipitate can be controlled by the composition of the mixture, ratio, and concentration of the components, surface charge density, pH and vortex speed, etc.

The composition is derived from ECM, HA, collagen, and GAGS to support cell survival, proliferation, and/or differentiation. In embodiments, the GAG/HYP ratio of the precipitates is high and controllable within a range of 1:1 to 100:1, or 1:1 to 50:1. Also, the coprecipitates show high GAG retention ability (e.g. 10%-40% within 7 days), solving the problem of quickly GAG elution from solid meshwork. The coprecipitates show nano-size ‘beads’ like structure in SEM and ‘bottlebrush’ like structure in TEM, highly structurally mimic the native GAG-rich tissue such as native NP. The coprecipitates also show good biocompatibility, high cell viability (>93%), maintenance of cell phenotype in protein and gene level, and comparable mechanical properties to that of the native NP. In addition, the coprecipitates promote the stem cell differentiation into the chondrogenic and NP-like linage, maintain the scaffold volume by the GAG-water interaction, mimic the pericellular matrix of the native cartilage and NP, enhance the gene expression of the chondrogenic markers and discogenic markers. Last but not least, the coprecipitates show good biocompatibility in vivo that can integrate with the native cells and tissue and maintain the high GAG content. Moreover, the novel biomaterials also facilitates and promotes multiple differentiation potential of stem cells such as bone marrow mesenchymal stem cells, into lineages including but are not limited to chondrogenic and discogenic lineages.

In summary, the novel composition consists of high-density GAGS. The composition shows significantly high GAG incorporation and retention, biomimetic ultrastructure with nano-sized GAG ‘beads’ like and ‘bottlebrush’ like structure, good biocompatibility, and cell phenotype maintenance, highly structurally and functionally bio-mimic the native GAG-rich tissues. In addition, it promotes the stem cell differentiation such as chondrogenic and discogenic, maintains the scaffold volume by the GAG-water interaction, mimics the ultrastructure of pericellular matrix of the native tissue such as cartilage, NP, neurological tissue, synovial fluid, vitreous fluid, heart valves, lungs, liver, skin, and blood vessels, enhances the tissue specific gene expression such as chondrogenic and discogenic markers, good biocompatibility in vivo and maintain the high GAG content for at least one month in vivo, suggesting its potential application as a scaffold for GAG-rich tissue regeneration.

In one aspect of the invention, there is provided a composition comprising a glycosaminoglycan component, and one or more extracellular matrix (ECM) components forming a precipitate with the glycosaminoglycan component, wherein the precipitate has a glycosaminoglycan to hydroxyproline ratio from about 1:10 to about 100:1.

In some embodiments, the one or more ECM components are selected from the group consisting of collagen, hyaluronic acid, fibronectin, laminin, a core protein, a link protein, a peptide, a derivative thereof, a salt thereof, and a combination thereof. In a particular embodiment, the one or more ECM component comprises a core protein, a link protein, a peptide such as a self-assembled peptide (SAM), a synthetic peptide, a functional epitope of ECM component. The functional epitope of ECM component may be selected from Arg-Gly-Asp(RGD) peptide, Arg-Gly-Asp-Ser (RGDS), Gly-Arg-Gly-Asp-Ser-Pro (GRGDSP) for fibronectin, Gly-Phe-Pyrrolysine-Gly-Glu-Arg (GFOGER) for collagen, Ile-Lys-Val-Ala-Val (IKVAV) for laminin or a combination thereof.

In some embodiments, the one or more ECM components comprise collagen, hyaluronic acid, a derivative thereof, and/or a salt thereof, and at least one extracellular matrix component has a functional group reacting with the glycosaminoglycan component for forming the precipitate.

In some embodiments, the glycosaminoglycan component is selected from sulfated glycosaminoglycans, heparin/heparan sulfate, chondroitin sulfate, dermatan sulfate, keratan sulfate, a derivative thereof, or a combination thereof.

In some embodiments, the one or more ECM components have one or more amino groups for reacting with the glycosaminoglycan component to form the precipitate, and the one or more ECM components are positively charged or neutral.

In some embodiments, the ECM components comprise aminated collagen, and/or aminated hyaluronic acid, preferably comprise both aminated collagen and aminated hyaluronic acid. In embodiments where the collagen and/or HA is aminated, it is aminated by an amination reagent selected from ethylenediamine (EDA), tris(2-aminoethyl) amine (TAEA), L-arginine, metformin, or a polyamine, and preferably the amination reagent is ethylenediamine or tris(2-aminoethyl) amine.

In some embodiments, the precipitate comprises aCol(EDA)-GAG, aCol(TAEA)-GAG, aCol(EDA)-aHA-GAG, or aCol(TAEA)-aHA-GAG.

Hydroxyproline (HYP) is a marker for collagen. In some embodiments, the precipitate has the glycosaminoglycan to hydroxyproline (GAG/HYP) ratio from 1:1 to 100:1, from 1:1 to 50:1, preferably 5:1 or 27:1. In embodiments, the precipitate has a GAG/HYP ratio from 1:1 to 90:1, from 1:1:to 80:1, from 1:1 to 70:1, from 1:1 to 60:1, from 1:1 to 50:1, from 1:1 to 40:1, from 1:1 to 30:1, from 1:1 to 20:1, from 1:1 to 10:1, or from 1:1 to 5:1. In some embodiments, the precipitate has a GAG/HYP ratio of about 2:1, about 3:1, about 4:1, about 5:1, about 6:1, about 7:1, about 8:1, about 9:1, about 10:1.

In an embodiment, the precipitate has a GAG/HYP ratio from 1:1 to 10:1 which is suitable for differentiating mesenchymal stem cells (MSCs). In another embodiment, the precipitate has a GAG/HYP ratio of about 5:1 for creating an environment suitable for differentiating MSCs into cartilages cells or for promoting chondrogenic differentiation; or the precipitate has a GAG/HYP ratio of about 7:1 for creating an environment suitable for discogenic differentiation of MSCs, or for promoting discogenic differentiation of stem cells, i.e. differentiating into nucleus pulposus (NPC). The amount of the components in the composition may be modified according to various applications, e.g. for the purpose of mimicking native tissue matrix, with the desired GAG/HYP ratio. This is particularly advantageous in applications treating a subject having a tissue disorder.

In some embodiments, the precipitate shows GAG retention within 1 to 100 days, preferably for at least 7 days, for 1% to 99%, preferably 50%.

In some embodiments, the precipitate is in the form of small nano-sized ‘beads’ like structure, micro-scale aggregation, or ‘bottlebrush’ like structure. The precipitate can mimic the native tissue matrix.

In some embodiments, the precipitate has biocompatibility in terms of in vitro cell viability ranging from 50% to 99%, 70 to 99%, or 95%.

In some embodiments, the precipitate promotes and maintains cell phenotype, supports cell survival and cell proliferation, and/or mimics the mechanical properties of native GAG-rich tissue.

In some embodiments, the precipitate is capable of enhancing gene expression of a chondrogenic marker, such as Col2, ACAN, and Sox9, and/or gene expression of a discogenic marker, such as PAX1 and FOXF1.

In some embodiments, the precipitate has biocompatibility in vivo for integrating with the native cells and tissues.

In some embodiments, the precipitate is capable of maintaining a high GAG content for a range from 1 day to 100 days either in vitro or in vivo.

In another aspect of the invention, there is provided a method of making the composition as described herein. The method comprises the steps of:
(i) providing one or more ECM components, optionally the one or more ECM components are aminated by an amination reagent as described above;
(ii) purifying the one or more ECM components optionally by dialysis;
(iii) providing an aqueous solution of one or more ECM components;
(iv) mixing the aqueous solution of step (iii) with a glycosaminoglycan component to form a precipitate, optionally followed by shaking or vortexing; and
(v) collecting the precipitate;

wherein the precipitate has a glycosaminoglycan to hydroxyproline ratio from about 1:10 to about 100:1.

In some embodiments, the amination reagent is a cationic chemical comprising at least two primary amino groups.

In some embodiments, the amination reagent is ethylenediamine (EDA), Tris(2-aminoethyl) amine (TAEA), L-arginine, Metformin, polyamines, or a combination thereof.

In a further aspect, there is provided a method of treating a tissue disorder in a subject comprising administering the composition as disclosed herein to the subject, wherein the composition serves as a swelling agent and/or a volume filing agent for implanting into a GAG-rich tissue in the subject.

In some embodiments, the GAG-rich tissue is nucleus pulposus (NP) or cartilage.

In a still further aspect, there is provided a method of culturing a tissue with abundant GAG. The method comprises a step of providing the composition as disclosed herein as a substrate, a cell-free scaffold or a cell-microcarrier. The composition may be used in 3D culturing to maintain the physiologically relevant phenotype of the parenchymal cells in native tissues.

In some embodiments, the tissue is nucleus pulposus (NP) or cartilage.

In some embodiments, the parachymal cells are chondrocytes in cartilage.

In some embodiments, the parachymal cells are nucleus pulposus cells (NPCs) in NP.

In another aspect of the invention, there is provided a device comprising the composition as disclosed herein. In an embodiment, the device further comprises stem cells, or cells isolated from cartilage, bones and nucleus pulposus.

### BRIEF DESCRIPTION OF DRAWINGS

The patent or application file contains at least one drawing executed in color. Copies of this patent or patent application publication with color drawings will be provided by the Office upon request and payment of the necessary fee.

FIG. 1 is a schematic diagram illustrating the fabrication process of the GAG-rich composition.

FIG. 2 shows the collagen amination by EDA and characterization of aCol (EDA) and aCol(EDA)-GAG coprecipitates. A:Mechanism of amination reaction of aCol(EDA); B:FTIR spectra analysis of collagen(Col) and aCol(EDA); C:Zeta potential of Col and aCol(EDA); D:GAG /HYP ratio of Col-GAG and aCol(EDA)-GAG; E:SEM images of aCol(EDA)-GAG coprecipitates, E1:aCol(2 M EDA)-GAG at pH=7; E2:aCol(2 M EDA)-GAG at pH=3, scale bar=500 nm.

FIG. 3 shows the collagen amination by TAEA and characterization of aCol (TAEA) and aCol (TAEA)-GAG coprecipitates. A:Mechanism of amination reaction of aCol (TAEA); B:FTIR spectra analysis of Col and aCol(TAEA); C:Zeta potential of Col and aCol(TAEA); D:GAG/HYP ratio of Col-GAG and aCol(TAEA)-GAG; E:SEM images of aCol(TAEA)-GAG coprecipitates, E1:aCol(TAEA)-GAG at pH=7; E2:aCol(TAEA)-GAG at pH=3, scale bar=1 μm.

FIG. 4 shows the collagen amination by L-arginine and characterization of aCol (L-arginine) and aCol (L-arginine)-GAG coprecipitates. A:Mechanism of amination reaction of aCol (L-arginine); B:FTIR spectra analysis of Col, L-arginine and aCol(L-arginine) crosslinked by EDC or EDC/NHS; C:GAG/HYP ratio of Col-GAG and aCol(L-arginine)-GAG coprecipitates; D:SEM images of aCol(L-arginine)-GAG coprecipitate, scale bar=1 μm.

FIG. 5 shows the collagen amination by Metformin and characterization of aCol (Metformin) and aCol(Metformin)-GAG coprecipitates. A:Mechanism of amination reaction of aCol(Metformin); B:FTIR spectra analysis of Col, Metformin and aCol(Metformin) crosslinked by EDC or EDC/NHS; C:GAG/HYP ratio of Col-GAG and aCol(Metformin)-GAG; D:SEM images of aCol(Metformin)-GAG coprecipitate, scale bar=1 μm.

FIG. 6 shows HA amination by EDA and TAEA, and characterization of aHA and aCol-aHA-GAG coprecipitates. A:Mechanism of amination reaction of aHA(EDA); B:Mechanism of amination reaction of aHA(TAEA); C:FTIR spectra analysis of HA, aHA(EDA) crosslinked by EDC or EDC/NHS; D:FTIR spectra analysis of HA, aHA(TAEA) crosslinked by EDC or EDC/NHS; E and F:GAG/HYP ratio of different aCol-aHA-GAG coprecipitates.

FIG. 7 shows optimization of amination condition of aHA and aCol to form high GAG/HYP ratio coprecipitates, and characterization of FITR of aHA(TAEA), and GAG/HYP ratio of aCol-aHA-GAG coprecipitates. A:FTIR spectra analysis of HA, and aHA(TAEA) crosslinked by different EDC concentration and TAEA concentration; B:Zeta potential of HA, and aHA(TAEA) aminated by different TAEA concentration; C:Optimization of the GAG/HYP ratio of different Col-aHA(TAEA)-GAG and aCol-aHA(TAEA)-GAG Co-ppts formed different TAEA concentration; D:Optimization of the GAG/HYP ratio of different aCol-aHA(1 M TAEA)-GAG Co-ppts formed by aCol(EDA) and E:aCol(TAEA) with different EDA/TAEA concentration; F:Optimization of the GAG/HYP ratio of different aHA(TAEA)-aCol-GAG Co-ppts formed by different aCol(0.25 M EDA)/aHA(1 M TAEA) ratio.

FIG. 8 shows GAG release curve of bovine tail native NP, AF and IVD and different coprecipitates against time within 24 h (FIG. 8A) and 7 days (FIG. 8B) keep in medium at 37° C. Coprecipitates:Col-GAG coprecipitate; aCol(EDA)-GAG coprecipitate; aCol(TAEA)-GAG coprecipitate; Col-aHA-GAG coprecipitate; aCol(EDA)-aHA-GAG coprecipitate; aCol(TAEA)-aHA-GAG coprecipitate.

FIG. 9 shows changes in gross appearance of different coprecipitates against time within 14 days keep in medium at 37° C. A: Col-GAG coprecipitate; B: aCol(EDA)-GAG coprecipitate; C: aCol(TAEA)-GAG coprecipitate; D: Col-aHA-GAG coprecipitate; E:aCol(EDA)-aHA-GAG coprecipitate; F: aCol(TAEA)-aHA-GAG coprecipitate.

FIG. 10 shows SEM images, ‘beads’ like structure and diameter distribution of bovine tail native AF, native NP and different coprecipitates. A: native AF; B: native NP; C: Col-GAG coprecipitate; D: aCol(EDA)-GAG coprecipitate; E:aCol(TAEA)-GAG coprecipitate; F: Col-aHA-GAG coprecipitate; G: aCol(EDA)-aHA-GAG coprecipitate; H: aCol(TAEA)-aHA-GAG coprecipitate.

FIG. 11 shows TEM images, ‘bottlebrush’ like structure and diameter distribution of bovine tail native AF, native NP and different coprecipitates. A: native AF; B: native NP; C: Col-GAG coprecipitate; D: aCol(EDA)-GAG coprecipitate; E:aCol(TAEA)-GAG coprecipitate; F: Col-aHA-GAG coprecipitate; G: aCol(EDA)-aHA-GAG coprecipitate; H: aCol(TAEA)-aHA-GAG coprecipitate.

FIG. 12 shows the TEM images of aCol(EDA)-aHA(TAEA)-GAG with different ratio of aCol(EDA)/aHA. A-A2:aCol(EDA)-aHA-GAG with aCol(EDA)/aHA ratio 1:0.5; B-B2: 1:2 and C-C2:1:8. scale bar=10 μm for A-C, 1 μm for A1-C1, 200 nm for A2-C2.

FIG. 13 showsLive/dead staining of bNPCs encapsulated coprecipitates culture for 3 d, 7 d, d and 14 d. A: Col microsphere; B: Col-GAG coprecipitate; C: aCol(EDA)-GAG coprecipitate;

D: aCol(TAEA)-GAG coprecipitate; E: Col-aHA-GAG coprecipitate; F: aCol(EDA)-aHA-GAG coprecipitate; G: aCol(TAEA)-aHA-GAG coprecipitate, H: Cell viability of bNPCs encapsulated in Co-ppts; scale bar=100 μm.

FIG. 14 shows H&E staining of bNPCs encapsulated coprecipitates culture for 3 d, 7 d, 10 d and 14 d. A: Native NP; B: Col microsphere; C: Col-GAG coprecipitate; D: aCol(EDA)-GAG coprecipitate; E:aCol(TAEA)-GAG coprecipitate; F: Col-aHA-GAG coprecipitate; G: aCol(EDA)-aHA-GAG coprecipitate; H: aCol(TAEA)-aHA-GAG coprecipitate, scale bar=100 μm.

FIG. 15 shows Safranin-O staining of bNPCs encapsulated coprecipitates culture for 3 d, 7 d, 10 d and 14 d. A: Native NP; B: Col microsphere; C: Col-GAG coprecipitate; D: aCol(EDA)-GAG coprecipitate; E:aCol(TAEA)-GAG coprecipitate; F: Col-aHA-GAG coprecipitate; G: aCol(EDA)-aHA-GAG coprecipitate; H: aCol(TAEA)-aHA-GAG coprecipitate, scale bar=100 μm.

FIG. 16 shows IHC staining of HA in bNPCs encapsulated coprecipitates culture for 3 d, 7 d, 10 d and 14 d. A: Native NP; B: Col microsphere; C: Col-GAG coprecipitate; D: aCol(EDA)-GAG coprecipitate; E:aCol(TAEA)-GAG coprecipitate; F: Col-aHA-GAG coprecipitate; G: aCol(EDA)-aHA-GAG coprecipitate; H: aCol(TAEA)-aHA-GAG coprecipitate, scale bar=100 μm.

FIG. 17 shows F-actin staining of bNPCs encapsulated coprecipitates culture for 3 d, 7 d, d and 14 d. A: Native NP; B: Col microsphere; C: Col-GAG coprecipitate; D: aCol(EDA)-GAG coprecipitate; E:aCol(TAEA)-GAG coprecipitate; F: Col-aHA-GAG coprecipitate; G: aCol(EDA)-aHA-GAG coprecipitate; H: aCol(TAEA)-aHA-GAG coprecipitate, scale bar=100 μm.

FIG. 18 shows SNAP25 staining of bNPCs encapsulated coprecipitates culture for 3 d, 7 d, d and 14 d. A: Native NP; B: Col microsphere; C: Col-GAG coprecipitate; D: aCol(EDA)-GAG coprecipitate; E:aCol(TAEA)-GAG coprecipitate; F: Col-aHA-GAG coprecipitate; G: aCol(EDA)-aHA-GAG coprecipitate; H: aCol(TAEA)-aHA-GAG coprecipitate, scale bar=100 μm.

FIG. 19 shows KRT8 staining of bNPCs encapsulated coprecipitates culture for 3 d, 7 d, 10 d and 14 d. A: Native NP; B: Col microsphere; C: Col-GAG coprecipitate; D: aCol(EDA)-GAG coprecipitate; E:aCol(TAEA)-GAG coprecipitate; F: Col-aHA-GAG coprecipitate; G:

aCol(EDA)-aHA-GAG coprecipitate; H: aCol(TAEA)-aHA-GAG coprecipitate, scale bar=100 μm.

FIG. 20 shows qRT-PCR results showing the expression bNPCs marker genes in Col and Co-ppts. Co-ppts (Co-ppt I Col-GAG, Co-ppt II aCol(EDA)-GAG, Co-ppt III aCol(TAEA)-GAG, Co-ppt IV Col-aHA-GAG, Co-ppt V aCol(EDA)-aHA-GAG, Co-ppt VI aCol(TAEA)-aHA-GAG). A: COL2, B: ACAN; C: SNAP25; D: KRT8; E: CDH2; F: SOSTDC1; The expression levels were normalized to GAPDH and monolayer, which was given a value of 1 for each gene.

FIG. 21 shows elastic modulus of acellular Co-ppts and bNPCs encapsulated Co-ppts. A: Illustration of the microplate compression method; B: Phase-contrast image of microplate compression; C: Displacement-time curve of the sample during the step-change in microplate compression method; D: Reduced elastic modulus of acellular Co-ppts and bNPCs encapsulated Co-ppts; Data are expressed as mean±2SE of n=3-12 experiments.

FIG. 22 shows the gross appearance of hMSCs encapsulated microspheres and osteogenic differentiation for 7, 14, and 21 days. A1-A3: Col; B1-B3: Col-GAG; C1-C₃:aCol-GAG; D1-D3:

aCol-aHA-GAG, E: Diameter of microspheres overtime change of size of the scaffolds, scale bar=500 μm.

FIG. 23 shows H&E staining of hMSCs encapsulated microspheres and osteogenic differentiation for 7, 14, and 21 days. A1-A3: Col; B1-B3: Col-GAG; C1-C₃:aCol-GAG; D1-D3:aCol-aHA-GAG, scale bar=200 μm.

FIG. 24 shows Von Kossa staining of hMSCs encapsulated microspheres and osteogenic differentiation for 7, 14, and 21 days. A1-A3: Col; B1-B3: Col-GAG; C1-C₃:aCol-GAG; D1-D3:aCol-aHA-GAG, scale bar=200 μm.

FIG. 25 shows SEM images showing the ultrastructure of hMSCs encapsulated scaffold and osteogenic differentiation for 21 days. A1 and A2: Col; B1 and B2: Col-GAG; C1-C2:aCol-GAG; D1 and D2:aCol-aHA-GAG, scale bar=500 nm for A1-D1, 2 μm for A2-D2.

FIG. 26 shows EDX mapping, map sum spectrum, element analysis, and Ca/P ratio showing the ultrastructure of hMSCs encapsulated scaffold and osteogenic differentiation for 21 days. A1 and A2: Col; B1 and B2: Col-GAG; C1-C2:aCol-GAG; D1 and D2:aCol-aHA-GAG, E: Weight percentage of Ca and P deposition in 4 scaffolds, F: Ca/P molar ratio of 4 scaffolds and compared to that in the native bone.

FIG. 27 shows expression of osteogenic markers in hMSCs encapsulated in different scaffolds osteogenic differentiation for 7, 14, and 21 days. The expression of A: BMP2; B: RUNX2; C: ALP, data are presented as mean±2SE.

FIG. 28 shows the gross appearance of hMSCs encapsulated microspheres and chondrogenic differentiation for 7, 14, and 28 days. A1-A3: Col; B1-B3: Col-GAG; C1-C3:aCol-GAG; D1-D3:aCol-aHA-GAG, E: Diameter of microspheres overtime change of size of the scaffolds, scale bar=500 or 200 μm.

FIG. 29 shows the H&E staining of hMSCs encapsulated microspheres and chondrogenic differentiation for 7, 14, and 28 days. A1-A3: Col; B1-B3: Col-GAG; C1-C3:aCol-GAG; D1-D3:aCol-aHA-GAG, scale bar=200 μm.

FIG. 30 shows the Safranin 0 staining showing the GAG positive area of hMSCs encapsulated microspheres and chondrogenic differentiation for 7, 14, and 28 days.A1-A3: Col; B1-B3: Col-GAG; C1-C₃:aCol-GAG; D1-D3:aCol-aHA-GAG, scale bar=200 μm.

FIG. 31 shows TEM images showing the ultrastructure of cells and pericellular matrix of hMSCs encapsulated scaffold and chondrogenic differentiation for 28 days. A1 and A2: Native cartilage; B1 and B2: Col; C1 and C2: Col-GAG; D1 and D2:aCol-GAG; E1 and E2:aCol-aHA-GAG, scale bar=2 μm for A1-E1, 500 nm for A2-E2.

FIG. 32 shows gene Expression of chondrogenic markers in hMSCs encapsulated in different scaffolds and chondrogenic differentiation for 7, 14, and 28 days. The gene expression of A: SOX9; B: ACAN; C: Col2, data are presented as mean±2SE.

FIG. 33 shows the gross appearance of hMSCs encapsulated microspheres and discogenic differentiation for 7, 14, and 28 days. A1-A3: Col; B1-B3: Col-GAG; C1-C3:aCol-GAG; D1-D3:aCol-aHA-GAG, E: Diameter of microspheres overtime change of size of the scaffolds, scale bar=500 or 200 μm.

FIG. 34 shows the H&E staining of hMSCs encapsulated microspheres and discogenic differentiation for 7, 14, and 28 days. A1-A3: Col; B1-B3: Col-GAG; C1-C₃:aCol-GAG; D1-D3:aCol-aHA-GAG, scale bar=200 μm.

FIG. 35 shows the Safranin 0 staining showing the GAG positive region of hMSCs encapsulated microspheres and discogenic differentiation for 7, 14, and 28 days. A1-A3: Col; B1-B3: Col-GAG; C1-C₃:aCol-GAG; D1-D3:aCol-aHA-GAG, scale bar=200 μm.

FIG. 36 shows TEM images showing the ultrastructure of cells and pericellular matrix of hMSCs encapsulated scaffold and discogenic differentiation for 28 days. A1 and A2: Bovine NP; B1 and B2: Col; C1 and C2: Col-GAG; D1 and D2:aCol-GAG; E1 and E2:aCol-aHA-GAG, scale bar=2 μm for A1-E1, 500 nm for A2-E2.

FIG. 37 shows gene Expression of NPC markers of hMSCs encapsulated in different scaffolds and discogenic differentiation for 7, 14, and 21 days. The expression of A: Col2; B: ACAN; C: PAX1, D: FOXF1, data are presented as mean±2SE.

FIG. 38 shows gross appearance of different acellular scaffolds after subcutaneous implantation in a nude mouse for 1 month. A1-A2: Col (A2 is not shown because the collagen scaffold is completely resorbed and could not be retrieved); B1-B2: Col-GAG; C1-C2:aCol-GAG; D1-D2: Col-aHA-GAG; E1-E2:aCol-aHA-GAG, scale bar=5 mm.

FIG. 39 shows H&E staining of different acellular scaffolds after subcutaneous implantation in a nude mouse for 1 month. A1-A2: Col; B1-B2: Col-GAG; C1-C2:aCol-GAG; D1-D2: Col-aHA-GAG; E1-E2:aCol-aHA-GAG, scale bar=500 μm for A1-E1, 200 μm for A2-E2.

FIG. 40 shows Safranin 0 staining of different acellular scaffolds after subcutaneous implantation in a nude mouse for 1 month. A1-A2: Col; B1-B2: Col-GAG; C1-C2:aCol-GAG; D1-D2: Col-aHA-GAG; E1-E2:aCol-aHA-GAG, scale bar=500 μm for A1-E1, 200 μm for A2-E2.

FIG. 41 shows gross appearance and H&E staining of hMSCs-encapsulated scaffolds and discogenic differentiated for 28 days and then subcutaneous implantation in a nude mouse for 1 month. A1-A3: Col; B1-B3: Col-GAG; C1-C₃:aCol-GAG; D1-D3:aCol-aHA-GAG, scale bar=5 mm for A1-D1, 500 μm for A2-D2, 200 μm for A3-D3.

FIG. 42 shows Safranin 0 staining of hMSCs-encapsulated scaffolds and discogenic differentiated for 28 days and then subcutaneous implantation in a nude mouse for 1 month. A1-A2: Col; B1-B2: Col-GAG; C1-C2:aCol-GAG; D1-D2:aCol-aHA-GAG, scale bar=500 μm for A1-D1, 200 μm for A2-D2.

### DETAILED DESCRIPTION OF THE INVENTION

### Definitions

As used herein, the GAG-rich biomaterial refers to the formation of a nanofibrous scaffold with high GAGS density. GAGS refer to the heparin/heparan sulfate, chondroitin sulfate/dermatan sulfate, and keratan sulfate, or any combinations of these moieties.

As used herein, “ECM” refers to the extracellular matrix materials, and ECM component may be provided in pure, isolated, partially isolated, recombinant or synthetic form. ECM component includes but not limited to HA, collagen, fibronectin, laminin, a core protein, a link protein, as well as a peptide, a derivative thereof, a salt thereof, and/or a combination thereof. The peptide includes, but not limited to, a self-assembled peptide (SAM) and a synthetic peptide such as a functional epitope of ECM component. The functional epitope of ECM component includes, but not limited to, Arg-Gly-Asp(RGD) peptide, Arg-Gly-Asp-Ser (RGDS), Gly-Arg-Gly-Asp-S er-Pro (GRGDSP) for fibronectin, GFOGER for collagen, and IKVAV for laminin.

### Materials for Fabrication of GAG-Rich Composition

A. ECM Materials

The ECM component used for the composition must be able to provide support to the cells and interact with the cells to allow cell growth, permitting cell migration and penetration without introducing toxicity. The ECM component used can be collagen, such as type I, II, and III, or hyaluronic acid, hyaluronan, hyaluronic acid sodium salt from bovine vitreous humor, rooster comb, Streptococcus equi or streptococcus zooepidemicus, other ECM component such as fibronectin, laminin, a core protein, a link protein, and peptides include self-assembled peptides (SAM) and synthetic peptide such as functional epitopes of ECM including but not limited to Arg-Gly-Asp(RGD) peptide, Arg-Gly-Asp-Ser (RGDS), Gly-Arg-Gly-Asp-Ser-Pro (GRGDSP) for fibronectin, GFOGER for collagen, and IKVAV for laminin. The ECM component can be derived from either natural or synthetic sources, and it can be induced to solid form under specific conditions and support cell survival and growth. The ECM component can be produced from isolation or extraction from various animal sources, such as rat tail, porcine skin, bovine tendon, or human placenta.

The anionic ECM component, which can be a proteoglycan or GAG of different types, such as heparin/heparan sulfate, chondroitin sulfate/dermatan sulfate, and keratan sulfate. The aminated ECM components can offer a binding site to anionic GAGS similar to that the GAGS link to the hyaluronan chain in the native proteoglycan structure. These ECM components can form interaction in such a way that leads to a change in the components and structure of the composition such as the volume, ultra-structure, morphology, ECM density, GAG/HYP ratio, GAG retention ability, mechanical property and stability, biocompatibility, etc.

B. Chemical Modification Reagent

The chemical modification reaction refers to using chemical groups of ECM component that react with primary amines (—NH2) and introduce the positively charged amino groups to the ECM chain. The chemical groups of ECM component can react with the primary amines refer to the carboxylic groups. The amination reagents can be from diverse origins; in preferred embodiments, chemicals are abundant of amino groups and non-toxic include but not limited to ethylenediamine (EDA), Tris(2-aminoethyl) amine (TAEA), L-arginine, Metformin, polypeptide, and polyamines. The reaction can be induced and crosslinked by crosslinker EDC, EDC/NHS, or other reagents that has good biocompatibility. The resulting solution can be dialysis or centrifugation to remove the unreacted chemical reagents.

### Amination Modification

The chemical modification method includes exposing the species such as HA or collagen reacting with amination reagents in specific amination reagents, pH, molar ratio, concentration, temperature, crosslinker, the concentration of crosslinker, and reaction time. The amination reagents used are chemicals that contain amino groups such as ethylenediamine (EDA), Tris(2-aminoethyl) amine (TAEA), L-arginine, Metformin, and polyamines, etc. Crosslinker used include 1-ethyl-3(3-dimethylaminopropyl) carbodiimide (EDC) in DI water and EDC/N-Hydroxy succinimide (NHS) (1 mM-100 mM) in 2-(N-morpholino) ethane sulfonic acid (IVIES) buffer solution. ECMs, amination reagents, and crosslinker are mixed thoroughly. In addition, the unreacted amination chemicals can be removed by dialysis. Aminated ECMs are collected after complete dialysis removing the unreacted chemicals.

The conditions of different amination reagents are adjusted for maintaining the high positive charge of aminated ECM components. The liquid-form amination reagents are diluted by hydrochloric acid (HCl) at a concentration between 0.01 M to 8 M, preferably 6-8 M, and this process must be operated on ice as the dilution process is exothermic. pH and concentration of the amination reagents diluted in HCl is adjusted by HCl and DI water under a pH meter. Moreover, the solid-form amination reagents are dissolved in IVIES buffer.

The amination reaction process is initiated by controlling the temperature, the pH, the ratios of reactants, crosslinker of the liquid environment at the appropriate time. The temperature of the amination reaction is raised from 4° C. to 10° C., 20° C., 37° C., and preferably 4-20° C. The reaction environment is maintained at pH between 1 to 13, preferably between 5-6 for both liquid-form and solid-form amination reagents. The positive charge of the aminated ECM components can be increased by increasing the ratio of amination reagents to ECM components. The molar ratios of amination reagents to ECMs are between 1:1 to 5000:1, preferably 50:1 to 5000:1. The crosslinker is responsible for crosslinking the carboxylic groups of ECMs and amino amination reagents. The crosslinker of the reaction is EDC or EDC/NHS. The reaction time is controlled between 0.5 h to 24 h, preferably 2-16 h.

The aminated ECM components can be purified by a dialysis tube or high-speed centrifugation in a microtube with a dialysis membrane, preferably a dialysis tube. The aminated ECM components are dialyzed against liquid such as DI water, phosphate-buffered saline, and dilute acetic acid solution for 2-4 days, and change fresh dialyzing solution 4 times/day. The aminated ECM components are stabilized by collecting the solution from the dialysis tube, keeping the solution at 4° C. or freeze-drying and storing it in −20° C.

The surface charge of the aminated ECM components can be controlled by at least one of the following parameters: composition, chemical groups of ECM components, the concentration of the ECM components, and amino group density, and concentration of amination reaction reagents. The ECM components suitable for amination include collagen, HA, and other ECM components, and the concentration of ECM components can be controlled at a range between about 0.01 mg/ml to 30 mg/ml. The amination reaction chemicals include ethylenediamine (EDA), Tris(2-aminoethyl) amine (TAEA), L-arginine, Metformin, and polyamines, etc. The concentration of amination reagents can be controlled at a range from 0.001 to 10 M, or preferably 0.25 M to 2.5 M for EDA, 0.15 M to 1 M for TAEA, and 0.1 M to 2 M for L-arginine and Metformin. Increasing the concentration of the amino group increases the positive charge of the aminated ECM components.

### Method of Making GAG-Rich Composition

The method of forming GAG-rich composition process includes mixing the components together, vortex and centrifugation. The GAG-rich coprecipitate can be fabricated by different aminated ECM components mixture. The surface charge density of ECM components depends on the amination reagent concentration during the amination process that ECM components reacted with a high concentration of amination reagents have higher GAGS incorporation. The size, GAGS retention property, and mechanical property of the composition can be controlled by the components of the mixture, ratio, and concentration of the components, surface charge density, pH, and vortex speed, etc.

The system for producing GAG-rich composition includes a unit for mixing the components for gelation of the coprecipitate; and a platform for collecting the coprecipitates. The aminated ECM components and anionic GAGS are mixed and evenly distributed throughout the solution before co-precipitation. The gelation process can be accelerated by shaking or vortex. The coprecipitates are collected after centrifugation and removing the supernatant.

The gel formation process of the composition is also initiated by controlling the temperature, the pH, and the aminated ECM components concentration. The gelation process of the GAG-rich coprecipitates is maintained at a temperature between 4° C. to 37° C., or more preferably between 20° to 37° C. The pH of the gel formation process is maintained from 1 to 13, preferably 7. The coprecipitates can be formed by mixing the aminated ECM components and anionic GAGS in a short time, for example, within a range between 10 s to 30 minutes depending on the aminated ECM components concentration. The gelation speed can be controlled as fast as immediately after the optional shaking or vortex, or raising the temperature of the mixture to 37° C., or increasing the concentration of aminated ECM components and GAGS. The diameter of the coprecipitates formed controlled at a range between 0.002 mm to 50 mm. The gelled coprecipitates are collected by the gravity action or centrifugation. The free aminated ECM components and GAGS are gentle flushing with a liquid such as culture medium, DI water, or phosphate-buffered saline.

The size, GAG/HYP ratio, GAG retention property, and mechanical properties of the composition can be controlled by at least one of the following parameters: composition and concentration of aminated ECMs, the combination of two or more aminated ECM components together, amination conditions of the aminated ECM components, the ratio of the aminated ECM components to GAGS, and ratio of two aminated ECM components. For example, the initial aminated ECM components concentration can be controlled at a range between 0.01 to 30 mg/ml, using two or more aminated ECM components, preferably aHA and aCol mixed, the ratio of the aminated ECM components to GAGS can range from 1:10 to 10:1, preferably 1:2, the ratio of the aCol and aHA is range from 8:1 to 1:8, preferably 1:2-1:8, aHA are aminated by ethylenediamine (EDA), Tris(2-aminoethyl) amine (TAEA), L-arginine, Metformin, polypeptide, and polyamines, preferably aHA(TAEA) with TAEA concentration at a range of 0.5 M-1M. Similarly, the amination condition of aCol is reacted with ethylenediamine (EDA), Tris(2-aminoethyl) amine (TAEA), L-arginine, Metformin, polypeptide, and polyamines, preferably aCol(EDA) with EDA concentration at a range of 0.25 M-2.5 M and aCol(TAEA) with TAEA concentration at a range of 0.015 M-1M.

Properties of GAG-rich composition

The novel composition is fabricated by naturally occurring ECM component, HA, collagen, and GAGS, suggesting good biocompatibility that supports cell survival and proliferation. The GAG/HYP ratio of the aCol-aHA-GAG coprecipitates is high and controllable with the range of 3.5:1 to 39.1:1, significantly higher than existing scaffolds. Also, the aCol-aHA-GAG coprecipitate shows high GAG retention ability (20%-60%) within 24 h and 10%-40% within 7 days, solving the problem of quickly GAG elution from solid meshwork. The aCol-aHA-GAG coprecipitate shows nano-size ‘beads’ like structure in SEM and ‘bottlebrush’ like structure in TEM, highly structurally mimic the native GAG-rich tissue such as native NP. The aCol-aHA-GAG coprecipitate can be used for cell-free and cell-carrier scaffolds, and the shape, size, the orientation of the cell encapsulated aCol-aHA-GAG coprecipitates can be controlled. The aCol-aHA-GAG coprecipitate also shows good biocompatibility, high cell viability (>93%), maintenance of cell phenotype in protein and gene level, and comparable mechanical properties to that of the native NP. In addition, the aCol-aHA-GAG coprecipitate promotes the stem cell differentiation into the chondrogenic and NP-like linage, maintain the scaffold volume by the GAG-water interaction, mimic the pericellular matrix of the native cartilage and NP, enhance the gene expression of the chondrogenic markers and discogenic markers. Last but not least, the aCol-aHA-GAG coprecipitate shows good biocompatibility in vivo that can integrate with the native cells and tissue and maintain the high GAG content for at least one month.

The GAG/HYP ratio and GAG retention of the aCol-aHA-GAG coprecipitate is high and controllable by adjusting the TAEA concentration of aHA(TAEA) amination, EDA and TAEA concentration in aCol(EDA) and aCol(TAEA) amination, aCol/GAG ratio, and aCol/aHA ratio. By optimization, the GAG/HYP ratio can be controlled from 0 to 39.1:1. Also, the GAG retention ability can be controlled at a range from 20%-60% within 24 h and 10%-40% within 7 days release.

The ultrastructure of the aCol-aHA-GAG coprecipitates can be controlled by at least one of the following parameters: the composition, the aCol/aHA ratio, pH, culture temperature, and culture time. The ultrastructure of the coprecipitates are varied form with different components that aCol(EDA)-GAG and aCol(TAEA)-GAG showed nano-size (20-40 nm) ‘beads’ like structure while aCol(EDA)-aHA-GAG and aCol(TAEA)-aHA-GAG show both small nano-size (20-40 nm) ‘beads’ like and large aggregates(100 nm-500 nm) structure in SEM. Furthermore, aCol(EDA)-GAG and aCol(TAEA)-GAG show thin and tight fibers while the aCol(EDA)-aHA-GAG and aCol(TAEA)-aHA-GAG show large aggregates(100 nm-2 μm) and ‘bottlebrush’ like (50 nm-200 nm in length)structure in TEM. The ‘beads’ like and ‘bottlebrush’ like ultrastructure of the aCol-aHA-GAG coprecipitate are similar to that of the native NP, suggesting that these biomaterials highly structurally mimic the native GAG-rich tissue.

The aCol-aHA-GAG coprecipitate can be used for cell-carrier by mixing the aminated ECMs, GAGS, and cells together. The shape, size, GAG/HYP ratio, cell density, matrix density of the cell encapsulated coprecipitates can be controlled. Cells used in encapsulation can be isolated from GAG-rich tissues, such as native NP, cartilage, neurological tissue, and other tissues, from human or larger animals, such as bovine and sheep. The shape and size can be controlled by centrifuge and transfer to tubes with different shapes. The cell density can be controlled at 1E4-1×1E6 cells/mg aCol, preferably 5E4-5×1E6 cells/mg aCol. The coprecipitates showed high cell viability>93% and maintenance of bNPC phenotypes, such as SNAP25 and KRT8 in protein level and SNAP25, KRT8, CDH2, and SOSTDC1 in gene level. The elastic modulus of the acellular coprecipitates is 0.78-1.12 KPa, and that of the bNPCs-encapsulated coprecipitates is 10.54-12.38 KPa, which is comparable to the native NP (3.21 KPa) and AF (16.31 KPa).

The aCol-aHA-GAG coprecipitate can also be used for the hMSCs differentiation 3D culture system. The types of stem cells can be induced pluripotent stem cells (iPSCs), embryonic stem cells (ESCs), and adult stem cells, such as mesenchymal stem cells, hematopoietic stem cells, neural stem cells, epithelial stem cells, and skin stem cells. Specifically, the mesenchymal stem cells can be derived from different sources, such as bone marrow, fat (adipose tissue), amniotic fluid (the fluid surrounding a fetus), or umbilical cord tissue (Wharton's Jelly). Stem cells can be induced to differentiation into the chondrocytes or NPC-like linage by adding the induction medium containing growth factors, such as TGF-03 for chondrogenic differentiation and TGF-β1 and/or GDFS for discogenic differentiation. The cell density can be controlled at 1E4-1×1E6 cells/mg aCol, preferably 5E4-5×1E6 cells/mg aCol. Upon chondrogenic differentiation, the coprecipitates promote the stem cell differentiation into the chondrocytes-like cells, increase the scaffold volume by the GAG-water interaction, mimic the lacuna circle and the ‘nanobeads’-fiber ultrastructure of the native cartilage, and enhance the gene expression of the chondrogenic phenotypes, such as Col2, ACAN, and SOX9. Upon discogenic differentiation, the coprecipitates maintain their volume, mimic the lamella circle, small fibers, and the ‘brush’ structures as that of the native NP, and enhance the expression of discogenic markers, such as Col2, ACAN, PAX1, and FOXF1.

The aCol-aHA-GAG coprecipitate show good biocompatibility in vivo that can integrate with the native cells and tissue. The coprecipitates can be subcutaneously implanted in a mouse. The coprecipitates can be acellular or cell encapsulated. After 1-month post-implantation, the coprecipitates integrate with the native skin cells and tissues without any signs of inflammation or foreign body reaction. In addition, the high GAG content is maintained after 1 month.

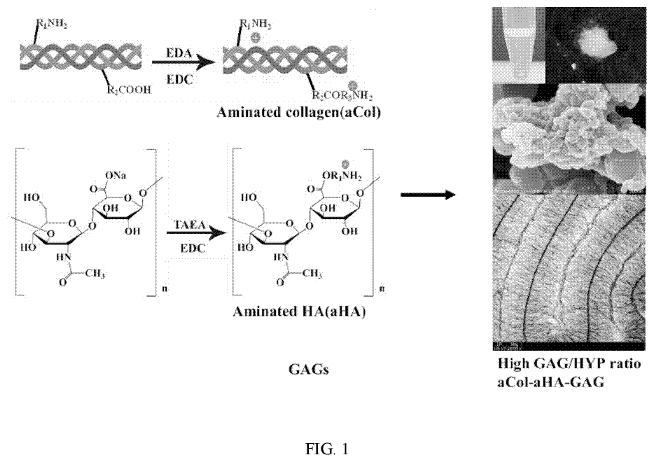

FIG. 1 shows a schematic illustration of producing the GAG-rich composition. The whole process comprises the amination reaction of ECMs, which consists of amination reactions for producing aCol and aHA, and then the aminated ECMs are mixed with anionic GAGS. The aCol-aHA-GAG coprecipitate with a high GAG/HYP ratio are formed in a microtube and collected at the bottom of the tube after centrifugation.

### Examples

The present invention will be further understood by reference to the following non-limiting examples.

### Example 1 Amination of Collagen

### Materials and Methods

Rat-tail collagen type I solution in acetic acid, which mainly consists of triple helical monomers, pH=6 ethylenediamine solution (EDA) solution diluted in 6 M HCl, and 2 M EDC solution in DI water were mixed together. All procedures were done in an ice-bath to prevent collagen gel formation. The mixture was maintained and reacted at room temperature overnight with 60 r/min shaking. To remove the unreacted reagent, the solution was then dialyzed using a dialysis tube against 0.023 M acetic acid at room temperature for 6 hours and then at 4° C. for 2 days, changed fresh 0.023 M acetic acid 4 times every day.

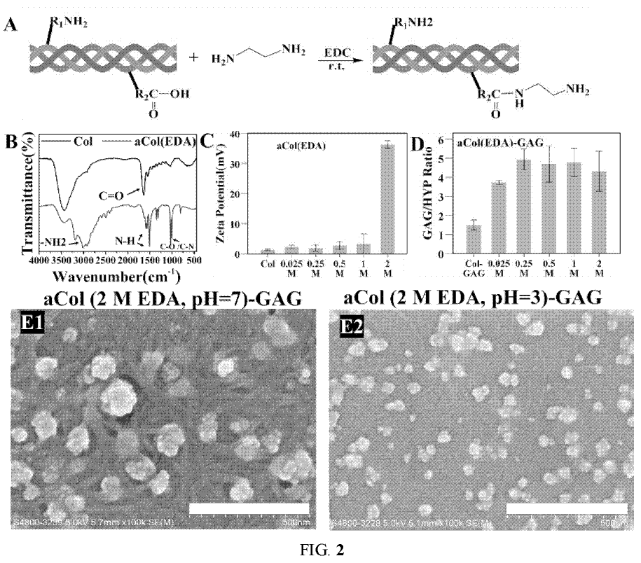

The carboxyl groups of collagen triple-helical chain were reacted with the amino groups of EDAs, which was shown in FIG. 2A. After amination, the stable amide bond was formed with the crosslinker EDC or EDC/NHS. The aCol (EDA) showed a positive charge as the EDA had two primary amino groups at both ends of the chain.

### Example 2 Controlling Parameters of the Amination of Collagen

### Materials and Methods

Rat-tail collagen solution type I was mixed and reacted with different amination reagents (EDA, TAEA, L-arginine, and Metformin) in the presence of different crosslinkers (EDC and EDC/NHS) as described in Example 1. The concentration of aminated reagents used in amination was controlled at 0.025 M to 2 M for EDA, 0.015 M to 1 M for TAEA, and 0.1 M to 2 M for L-arginine and Metformin. The crosslinker concentration was controlled at 1 mM to 100 mM for EDC in DI water and 1 mM to 100 mM for EDC/NHS in 0.5 M MES buffer. The pH during the reaction was adjusted to a range from 1 to 10. The mixture was reacted under 4° C. to 20° C. overnight. The aCol under different conditions were collected after removing the unreacted reagents by dialysis.

1 mg Col and aCol with different amination reagents were freeze-dried and added to 200 mg dry potassium bromide. Mixed thoroughly in a mortar and pressed into tablets by tablet press. The chemical structure change during amination was directly analyzed by Perkin-Elmer spectrum-100 FTIR spectrometer (Perkin-Elmer Instruments, USA) with a universal ATR (attenuated total reflectance) sampling accessory. The sample was scanned 16 times, and the FTIR spectrum was recorded in the range of 4000 cm⁻¹ to 450 cm⁻¹ with a resolution of 2 cm⁻¹.

Zeta potential was performed to detect the surface charge of Col and aCol. 50 μl Col or aCol were added to quartz plate, and zeta potential was measured using Smoluchowski model under dynamic laser by the DelsaMax PRO light scattering Analyzer (Beckman Coulter).

### Results

The aCol with different amination reagents, reagents concentration, and crosslinkers were studied in detail. The mechanism of amination reaction of aCol with different reagents was shown in FIGS. 2A, 3A, 4A, and 5A, respectively. The positive charge density was directly related to the amination reagents type and concentration, suggesting that these parameters can be used to control the final surface charge of the aCol.

FTIR was used to investigate the structural variation during the amination process. As shown in FIGS. 2B, 3B, 4B, and 5B, in the spectrum of collagen, it showed characteristic absorption peaks of —OH stretching vibration, C═O stretching, N—H bending, C—N stretching, and amide II, the deformation vibration of N—H and C—O/C—N stretching, respectively (Zhou, Yang et al. 2012, Jana, Mitra et al. 2016). After amination, the absorption intensity of C═O was weakening, which may be induced by —COOH reacting with the amino group and forming a new amide peak. The amino group-related characteristic absorption peaks were all showed high intensity (FIG. 2B, 3B, 4B, 5B). Without any doubt, the wavenumber and intensity of absorption peaks were different as the amination reagents changed to EDA, TAEA, L-arginine, and Metformin. FIG. 2C shows the zeta potential of aCol aminated by different EDA concentrations. Collagen was about neutral charge while aCol was positively charged with amino group incorporation. Furthermore, the positive charge was dose-defendant. Higher EDA concentration showed a higher positive charge.

### Example 3 Amination of Hyaluronic Acid

### Materials and Methods

Hyaluronic acid (HA) sodium salt was aminated using the same method as aCol in example 1. HA powder dissolved in DI water with different HA densities: 1 mg/ml to 4 mg/ml and used for the production of aHA with different amination reagents: EDA, TAEA. Similarly, EDA concentration was controlled at a range from 0.25 to 2 M, and that of TAEA was 0.15 M to 1 M. HA solution, EDA or TAEA, and crosslinker mixed together and reacted overnight at room temperature. The aHA was obtained after dialysis against DI water. FTIR and zeta potential were also used to investigate the changing of chemical bonds and surface charge during the amination process.

### Results

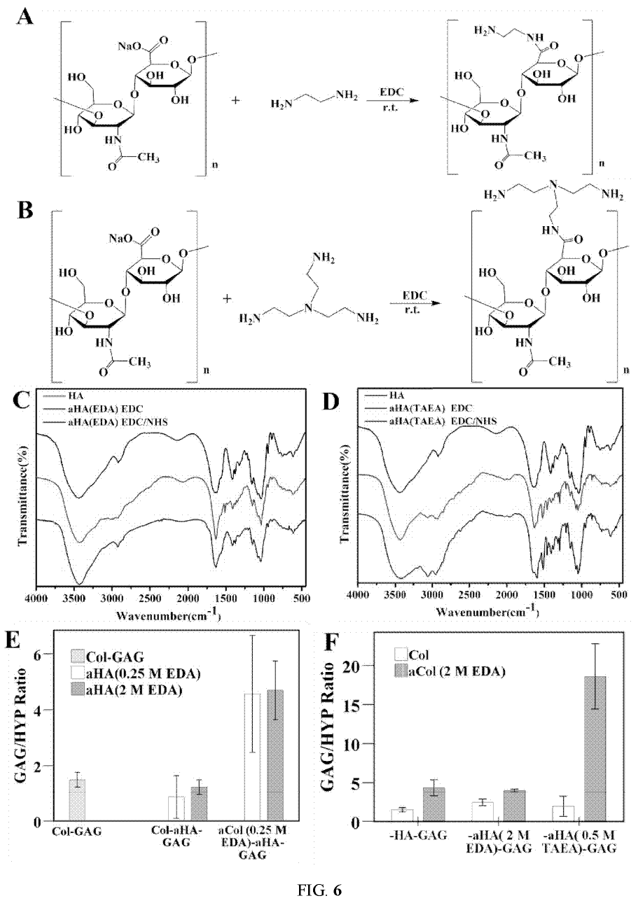

Hyaluronic acid is a polymer of disaccharides and is composed of repeated D-glucuronic acid and N-acetyl-D-glucosamine structures. Hence, HA is abundant in carboxyl groups that can react with the amino groups. As shown in FIGS. 6A and 6B, the repeated carboxyl groups were reacted with EDA or TAEA, resulting in aHA.

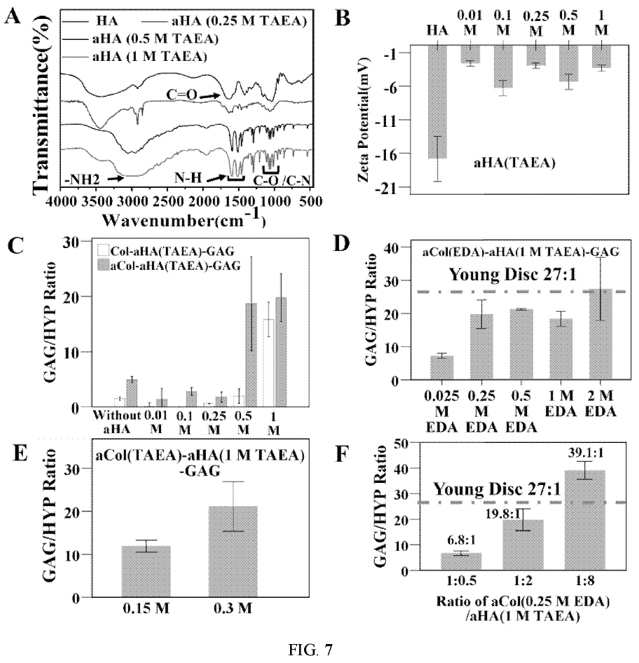

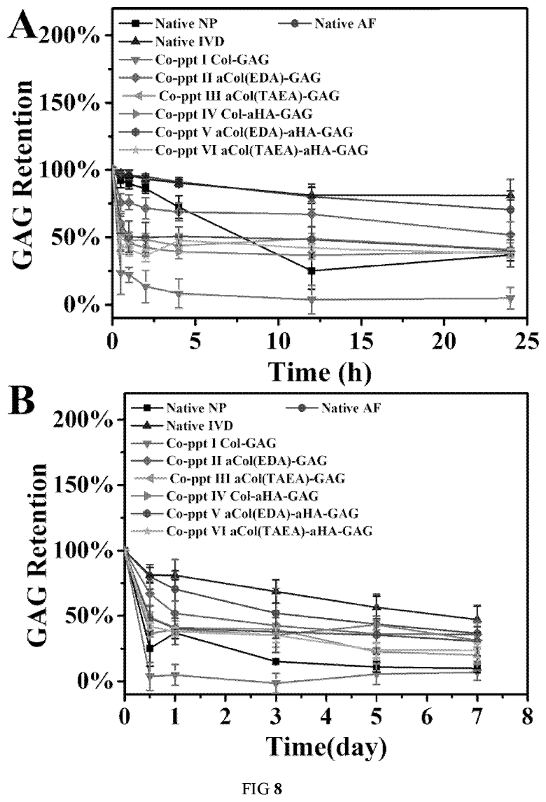

The FTIR spectra of HA and aHA were shown in FIGS. 6C, 6D, and 7A. After amination, the absorption of —C═O groups in HA was weakening, which may be induced by —COOH reacting with the amino group. The characteristic absorption peaks such as NH2 stretching vibration, N—H binding, C—O/C—N binding were all showed enhanced intensity, suggesting the increased signal of amino groups (Liu, Xu et al. 2018). These results indicated that EDA and TAEA were crosslinked to HA by covalent crosslinking. With TAEA amination, the intensity of absorption peaks related to amino groups enhanced with the TAEA concentration increased (FIG. 7A). As shown in FIG. 7B, negative charge (−17 mV) was detected in HA, and it tends to decrease after amination. The net charge of aHA approached neutral.

### Example 4 Production of GAG-Rich Composition and Ultrastructural Characterization

### Materials and Methods

GAG solution was prepared by dissolving chondroitin-6-sulfate from shark cartilage in DI water. aCol, either with or without aHA reacted by different amination reagents, was mixed with GAG (in excess) and vortex for 1 min. The coprecipitates were collected by centrifugation at 16100 g for 2 mins.

The coprecipitates after three times rinsing with DI water were solubilized by 200 μl 0.6 U papain solution at pH 6.5 containing 50 mM phosphate buffer (PB), 5 mM L-cysteine and 5 mM EDTA at 60° C. for overnight. The amount of GAG in the digested coprecipitate samples was diluted and detected by the dimethyl methylene blue (DMMB) method (Barbosa, Garcia et al. 2003). Briefly, 100 μl diluted samples were mixed with 1 ml 0.9% (w/v) DMMB solution and shaking the mixture on a shaker for 30 mins. The DMMB-GAGs complexes were collected by centrifugation at 14000 g for 10 mins and dissolved into the complex dissociation reagent. Absorbance at 656 nm of samples and standards were measured under a microplate reader. GAGS were quantified by a calibration curve of chondroitin sulfate standard between 1.25 and 40 pg/ml. Part of the digested samples was acidified with hydrochloric acid and hydrolyzed in a hydrolysis tube at a 120° C. heater for 4 h treatment. The hydrolyzed samples were neutralized to pH 6-7, and HYP content was measured by the chloramine T-dimethylaminobenzaldehyde (DMAB) method (Woessner 1961). HYP content was quantified using a calibration curve between 2.5 and 400 pg/ml, and the absorbance of the samples were detected at 557 nm. The GAG/HYP ratio was calculated by the GAG and HYP content of the same sample.

Scanning electron microscopy (SEM) was used to measure the ultrastructure of the co-precipitates immediately after fabrication (pH=3, pH=7) (Chan, Hui et al. 2007). Coprecipitates were prepared by mixing the aCol and GAGS with or without aHA. NaOH was added to aCol to give a final solution with neutral pH before collection by 16000 g 2 mins centrifugation. Samples were rinsed with DI water three times to remove free GAGS, fixed with 4% Paraformaldehyde (PFA) at 4° C. for an overnight treatment, and dehydrated using gradient ethanol (10%, 30%, 50%, 70%, 90%, 95% and 100%, 15 mins each conc.). And then, samples were dried by critical point drying and fractured to reveal the internal structure of the coprecipitates, sputter-coated by gold, and imaged using field emission SEM (S-4800, Hitachi, Tokyo).

### Results

aCol-GAG

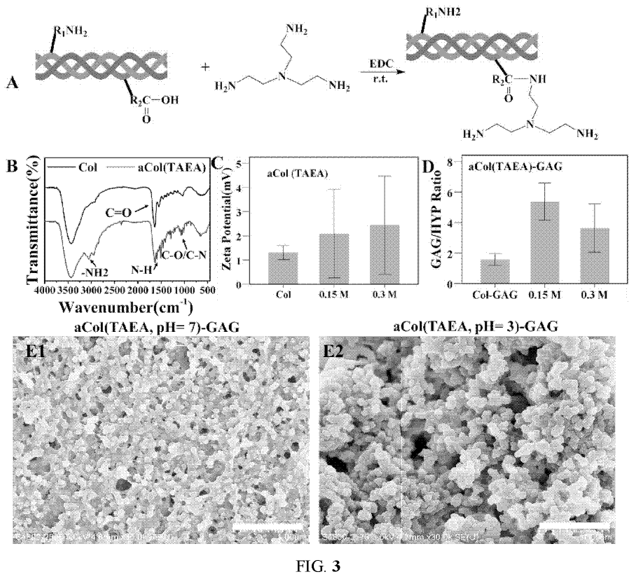

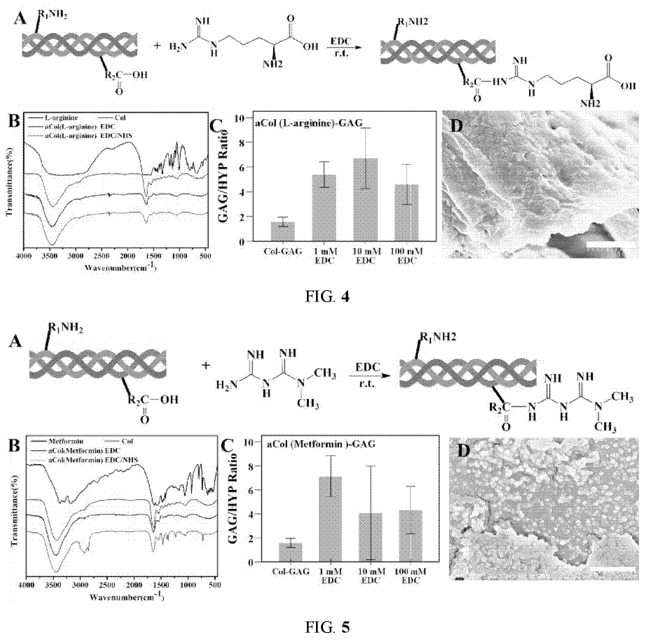

FIG. 2D, 3C, 4C, 5C showed a statistically significant increase of GAG content in aCol-GAG groups compared to that of Col-GAG. FIG. 2D showed the change of GAG content upon amination of various concentration of EDA (0.025 M to 2 M). The GAG/HYP ratio was increased from ˜2.3:1 to 3.7:1-4.9:1, and aCol(EDA)-GAG with different concentrations of EDA showed no significant difference in the GAG content as all the EDA concentration was excess to Col during amination. With TAEA amination, the GAG/HYP ratio was 3.6:1-5.4:1, similar to the aCol(EDA)-GAG. (FIG. 3C). As for solid-form amination reagents, L-arginine and Metformin were also used in amination. With 1 M L-arginine amination, the GAG/HYP ratio of aCol (L-arginine) increased to 4.5:1-6:1 crosslinked by 1 mM to 100 mM EDC (FIG. 4C). However, aCol (L-arginine) and aCol(metformin) crosslinked by EDC/NHS could not reconstitute into coprecipitate with GAGS because it may be formed NHS-intermediate, a highly hydrophilic substance and rapidly soluble in water. The GAG/HYP ratio of aCol(metformin) crosslinked by EDC was increased from 4:1 to 7:1 (FIG. 5C). These results showed that the amination significantly increased the GAG/HYP ratio to 3.6:1 to 7:1, suggesting the aCol has high GAG incorporation ability.

### SEM

The ultrafine fibrous structure of different aCol-GAG coprecipitates immediately after fabrication as shown in FIG. 2E,3E,4D,5D. In the aCol (EDA)-GAG group, abundant nano-sized ‘beads’ like structures with a diameter of about 100-200 nm were found in the bulk of samples, no matter under pH 7 or 3. However, the morphology of acidic conditions was different from that of under the pH 7 that occasional fibers underneath were observed at pH 7 (FIG. 2E). In the aCol(TAEA)-GAG group, the scaffold showed thick and aggregated fibrous collagen structures intercalating with ‘beads’ like structure both at pH 7 and 3(FIG. 3E). In the aCol(L-arginine)-GAG scaffold, a small amount of the ‘beads’ like substances was found in the tight fiber structure (FIG. 4D). In aCol(Metformin)-GAG scaffold, there were also the same nano-sized ‘beads’ like substances observed(FIG. 5D).

aHA-aCol-GAG

To further increase the GAG content, aHA was also reacted and added to form the coprecipitates. The repeated carboxylic groups reacted with amino groups can increase the positive charge density in coprecipitates. The incorporation of the aHA(EDA) was significantly increased the GAG content to 5.5:1-6:1 when aHA reaction crosslinked by EDC but not EDC/NHS(FIG. 6E). With the presence of the aCol, the GAG content was significantly increased compared to the Col-HA-GAG and Col-aHA-GAG. With the combined use of the aHA and aCol, an increasing trend was observed, especially the aCol-aHA-GAG, the GAG/HYP ratio increased to 18:1, suggesting the aHA (TAEA) cooperate with aCol showed high GAG incorporation ability.

### Example 5 Optimization of the GAG Content in GAG-Rich Composition and Characterization of Retention and Other Properties

### Materials and Methods

As mentioned in example 4, aCol-aHA (TAEA)-GAG showed the highest GAG/HYP ratio. The concentration of TAEA used in aHA amination was controlled between 0.01 M to 1 M, and that of EDC was controlled at 1 mM, 10 mM and 100 mM. In addition, various concentrations of EDA (0.025 M, 0.1 M, 0.125 M, 0.25 M, 0.5 M, 1 M, and 2 M), TAEA (0.15 M to 1 M) and aCol/aHA ratio (1:0.5-1:8) were used. The GAG/HYP ratio was used as a critical parameter and measured using the same method as described in example 4.

All coprecipitates, Col-GAG, aCol(EDA)-GAG, aCol(TAEA)-GAG, Col-aHA-GAG, aCol(EDA)-aHA-GAG, aCol(TAEA)-aHA-GAG were formed with 200 pg Col or aCol(EDA) or aCol(TAEA), 400 μg aHA, and 400 μg chondroitin-6-sulfate GAGS and immersed in 1 ml Dulbecco's Modified Eagle Medium(DMEM) Low Glucose (pH 7.2). GAGS were released in a shaking shaker (37° C., 180 rpm) over 7 days. At chosen time points (0.5, 1, 2, 4, 8, 12 and 24 h, 3, 5 and 7 days), 200 μl of the eluent in each sample was collected with the same volume of fresh DMEM-LG medium replacement, and the collected samples were stored in −20° C. for subsequent measurement of GAG content as described in example 4. The percentage of GAG retention in different coprecipitates at different time points was calculated. During the GAG release process, the gross appearance pictures of coprecipitates in different groups were taken at 0, 1, 3, 7, and 14 days. SEM was used to measure the ultra-structure of the coprecipitates immediately after fabrication. The diameter distribution of different samples was calculated by 100 random measurements of SEM images using Image-J software (National Institutes of Health, USA). Also, the GAG release curve and SEM images of native bovine tail annulus fibrosus (AF), nucleus pulposus (NP), and intervertebral disc (IVD) were measured and used as control.

Transmission electron microscopy (TEM) was used to measure the inner ultrastructure of the native bovine tail annulus fibrosus (AF), nucleus pulposus (NP), and coprecipitates immediately after fabrication. Coprecipitates were rinsed with DI water three times to remove the free GAGS, fixed with 4% PFA at 4° C. for overnight treatment. And then, samples were cut into 100 nm ultra-thin, spread to a TEM grid, and imaged using TEM (Philips CM100 TEM).

### Results

### GAG/HYP Ratio

The amination and formation conditions of aCol-aHA-GAG were optimized to form coprecipitates with a high GAG/HYP ratio. The GAG/HYP ratio increased as the concentration of TAEA used in aHA increased (FIG. 7C). The GAG/HYP ratio of Col-aHA(0.25 M TAEA)-GAG and aCol(0.25 M EDA)-aHA(0.25 M TAEA)-GAG were same as that of the Col-GAG, about 2:1, while aCol(0.25M EDA)-aHA(0.5 M TAEA)-GAG group showed increased GAG/HYP ratio, the With 1 M TAEA, the GAG/HYP ratio were 16:1 and 18:1 for Col-aHA (1 M TAEA)-GAG and aCol(0.25 M EDA)-aHA(1 M TAEA)-GAG, respectively (FIG. 7D). With aHA(1 M TAEA), the GAG content showed a significantly increase when the amination concentration of EDA or TAEA in aCol(EDA) and aCol(TAEA) increased(FIG. 7E, F). The GAG/HYP ratio was increased up to 27.4:1 in aCol(2 M EDA)-aHA-GAG, and that gradually decreased to 50%(12.3:1) in aCol(0.125 M EDA)-aHA-GAG, suggesting the concentration of EDA in aCol(EDA) should be higher than 0.125 M EDA. The same trend was observed in the concentration of TAEA. Furthermore, the GAG/HYP ratio of the Co-ppts significantly increased as the content of aHA(1 M TAEA) increased from 0.5 to 8 (FIG. 7G). The GAG/HYP ratio reached up to 39.1:1 when the ratio of aCol(0.25 M EDA)/aHA(1 M TAEA) was 1:8. Among all groups, aCol(2 M EDA)-aHA(1 M TAEA)-GAG with aCol/aHA ratio at 1:2 showed optimal performance in incorporating GAGS with GAG/HYP ratio 39.1:1.

### GAG Retention

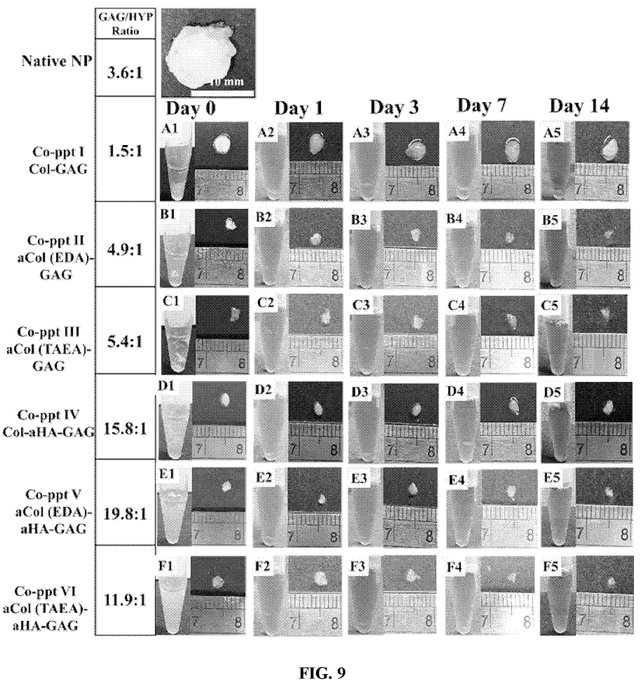

FIG. 8A-B showed the GAG release, and retention performance of the coprecipitates up to 24 h upon immersion in DMEM-LG medium. In native bovine tail NP, the GAG released rapidly without the protection of outer AF, only 40% GAG left within 24 h, and almost all GAG released after 7 days, which significantly less than aCol(EDA)-GAG, aCol(TAEA)-GAG, aCol(EDA)-aHA-GAG and aCol(TAEA)-aHA-GAG groups, suggesting these coprecipitates high GAG retention ability. The outer AF and unbroken IVD showed high GAG retention properties, indicating that the inner NP should be protected by the outer AF. In the Col-GAG group, almost all of the GAGS were released within the first 8 h (only<2% retained), and the percentage retained was significantly less than in the aCol(EDA)-GAG, aCol(TAEA)-GAG, aCol(EDA)-aHA-GAG and aCol(TAEA)-aHA-GAG group within the first 24 h. This trend was last until 7 days release.

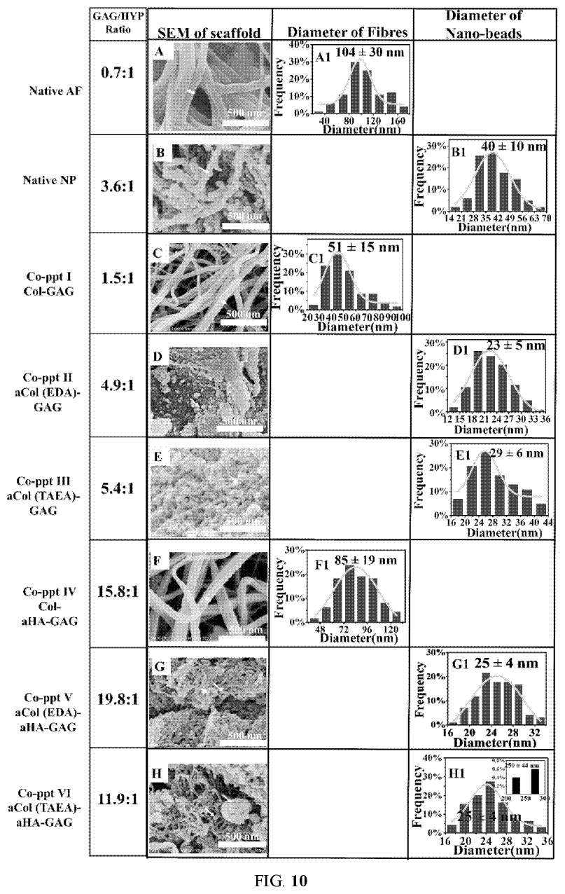

FIG. 9 showed the gross appearance pictures of coprecipitates in different groups at certain time points. With the same input amount of the aHA, Col or aCol, and GAG, the aCol(EDA)-GAG, aCol(TAEA)-GAG, aCol(EDA)-aHA-GAG and aCol(TAEA)-aHA-GAG groups formed smaller coprecipitates compared to the Col-GAG and Col-aHA-GAG groups, the high surface charge induced increased the interaction between aCol and GAGS and lead to tight morphology. Within 14 days of immersing in DMEM-LG, the size of Col-GAG and Col-aHA-GAG showed no significant difference from the beginning to 14 days while size reduction was observed in aCol(EDA)-GAG, aCol(TAEA)-GAG, aCol(EDA)-aHA-GAG and aCol(TAEA)-aHA-GAG groups.

### SEM

The ultrafine fibrous structure of native AF, native NP, and coprecipitates immediately after fabrication as shown in FIG. 10. In native AF, large collagen fibers (104 nm) with obvious D-bands were observed. Nano-sized ‘beads’ like structure (40 nm) was distributed on the fiber of native NP. In the Col-GAG group, long and thickened fibers with a diameter of 51 nm and obvious D-band were observed. Abundant Nano-sized ‘beads’ like structures were found in the aCol(EDA)-GAG(23 nm) and aCol(TAEA)-GAG(29 nm) group. The long fiber structure of Col-aHA-GAG was similar to that of Col-GAG, consisting of 85 nm collagen fibers with D-band in the same shape and diameter, and the difference between the two groups was that the increased ‘beads’ like structure and fiber diameter were observed in Col-aHA-GAG. Similarly, aCol(EDA)-aHA-GAG and aCol(TAEA)-aHA-GAG groups were all showed large ‘bead’ like structures(100 nm-500 nm) in which were a lot of small(20-30 nm) nano-sized ‘beads’ like structures, suggesting aCol(EDA)-aHA-GAG and aCol(TAEA)-aHA-GAG scaffolds incorporated more GAGS into the solid meshwork. Among all the coprecipitates, Col-GAG and Col-aHA-GAG groups showed similar structure as native AF while aCol(EDA)-GAG, aCol(TAEA)-GAG, aCol(EDA)-aHA-GAG and aCol(TAEA)-aHA-GAG groups showed similar ‘beads’ like GAG structure as native NP.

### TEM

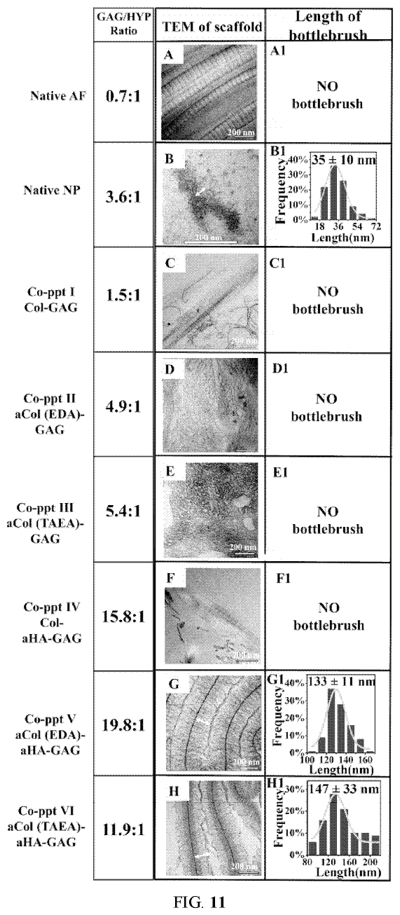

The ultrastructure of native AF, native NP, and coprecipitates were also verified by TEM and shown in FIG. 11. In native AF, collagen fibers with D-bands were observed. Nano-sized ‘beads’ like structure and fibers were distributed on the fiber of native NP. In addition, a small ‘bottlebrush’ structure (35 nm) was shown in the native NP. Fibers with large pores were shown in the Col-GAG and Col-aHA-GAG groups. The aCol(EDA)-GAG and aCol(TAEA)-GAG coprecipitates consisted of compactly arranged fibers due to the positive charge of aCol that high assembly interaction between aCol and GAG. Micron-scale granules were found in aCol(EDA)-aHA-GAG and aCol(TAEA)-aHA-GAG groups. Furthermore, long ‘bottlebrush’ structure was observed in aCol(EDA)-aHA-GAG (133 nm) and aCol(TAEA)-aHA-GAG (147 nm) groups. SEM and TEM results demonstrated that the Col-GAG and Col-aHA-GAG structurally bio-mimic the native AF while aCol(EDA)-GAG, aCol(TAEA)-GAG, aCol(EDA)-aHA-GAG and aCol(TAEA)-aHA-GAG, especially the aCol(EDA)-aHA-GAG and aCol(TAEA)-aHA-GAG can be used as a biomimetic scaffold for native NP.

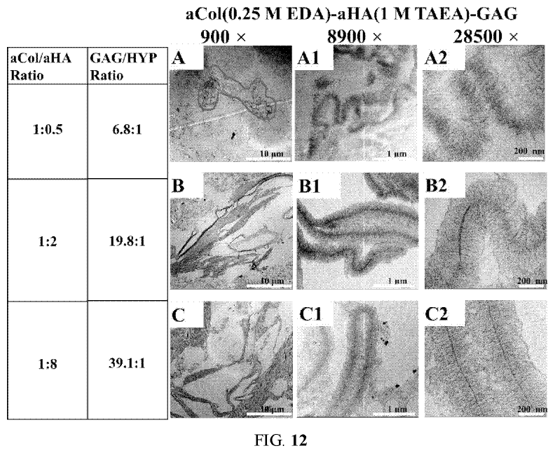

Furthermore, the ‘bottlebrush’ structures showed dose-dependent that the higher the aHA ratio, the more the ‘bottlebrush’ distributed in the TEM images (FIG. 12). When the aCol/aHA ratio was 1:0.5, the ‘bottlebrush’ structure was short. When the aCol/aHA ratio increased to 1:2 and 1:8, the distribution area of ‘bottlebrush’ structure increased, which was consistent with the GAG/HYP ratio data that higher aHA incorporation higher GAG content, indicating that the aHA was the backbone of the PGs structure, which recapitulates the ultrastructure of the native PGs.

### Example 6 Cell Viability, Cell Phenotype Maintenance and Histological Staining of Cell Encapsulated in GAG-Rich Composition

### Materials and Methods

Bovine nucleus pulpous cells(bNPCs) were extracted from the bovine tail and cultured in (Sigma-Aldrich) in Dulbecco's modified Eagle's medium with low-glucose (DMEM-LG) (Gibco, Thermo Fisher Scientific, Waltham, MA, USA) supplemented with 10% fetal bovine serum (FBS) (Gibco) and 1% penicillin/streptomycin (P/S) (Gibco) at 37° C. in a humidified incubator with 5% CO2. bNPCs of passage 2 were encapsulated in coprecipitates by thoroughly mixing with aHA, aCol, and GAGS. The bNPCs encapsulated coprecipitates after 3 days, 7 days, 10 days and 14 days culture were collected and stained with 1:1000 calcein-AM and ethidium homodimer-1 in PBS for 1 hour at 37° C.

Cell encapsulated coprecipitates on days 3, 7, 10, and 14 and native NP tissues were fixed in 4% PFA, and then were paraffin-embedded and sectioned at 10 μm thickness for subsequent histological Hematoxylin/eosin (H&E) staining and histochemical Safranin-O staining. The paraffin sections were de-waxed in a 70° C. oven and followed by two changes of xylene. The sections were serially rehydrated using gradient ethanol, and stained with hematoxylin for 12 minutes, counter-stained with eosin for 5 mins. The sections were eventually mounted with Depex after dehydration and xylene wash. To reveal areas rich in GAGS, Safranin-O staining was performed. The paraffin sections were treated in the same process as previously described. The bNPCs nuclei were first stained with hematoxylin QS for 5 minutes and then further stained with fast-green (FCF) solution to reveal non-collagen proteins, followed stained with 1.5% Safranin-O solution for 35 minutes and mounted with Depex. Images of samples stained with H&E and safranin 0 were captured using an inverted microscope.

Similarly, bNPCs encapsulated coprecipitates on days 3, 7, 10, and 14 were harvested and embedded into cryo-matrix, followed by cut into 15 μm sections. Samples were blocked in 1% BSA/10% normal goat serum/0.3 M glycine in 0.1% PBS-Tween for 1 h and then incubated with primary antibodies SNAP25 and KRT8 (in a humidified chamber for overnight at 4° C. And then incubated with secondary antibodies (Alexa Fluor 647-tagged anti-rabbit; Alexa Fluor 647-tagged anti-mouse) or Alex Fluor 488-taged F-actin and followed by mounting with mounting medium containing DAPI. The live/dead and immunofluorescence staining were acquired with confocal microscopy using a Leica SP8 Confocal Microscope and Imaging software Leica Application Suite (LAS) X.

To quantify of the expression of the phenotypic marker genes, the total RNA of bNPCs encapsulated in GAG-rich scaffolds was extracted by using the RNeasy Mini Kit (QIAGEN, Germany). Reverse transcription was carried out with a High-capacity Reverse Transcription Kit (Applied Biosystems). qPCR was then performed using the transcribed cDNA, the primers for Col2, ACAN, KRT8, SNAP25, CDH2, SOSTDC1, and GAPDH, the Power SYBR Green PCR Master Mix (Applied Biosystems), and a StepOnePlus Real-Time PCR System (Life Technologies). The primer sequences are listed in Table.1. The gene expression data were analyzed by the comparative CT method. Data were initially normalized to GAPDH, after which each gene was further normalized to the expression level of the monolayer culture.

TABLE 1
The primers used in the qRT-PCR.

| | Forward | Reverse |
|---|---|---|
| Gene | Primer (5′ to 3′) | Primer (5′ to 3′) |
| COL2 | CGGGTGAACGTGGAGAGACA | GTCCAGGGTTGCCATTGGAG |
| | (SEQ ID NO. 1) | (SEQ ID NO. 2) |
| KRT8 | ACCAGGAGCTCATGAATGTC | TCGCCCTCCAGCAGCTT |
| | AA | (SEQ ID NO. 4) |
| | (SEQ ID NO. 3) | |
| SNAP25 | GGCTTCATCCGCAGGGTAA | GCTCCAGGTTTTCATCCATT |
| | (SEQ ID NO. 5) | TC |
| | | (SEQ ID NO. 6) |
| CDH2 | GCCATCAAGCCAGTTGGAA | TGCAGATCGAACCGGGTACT |
| | (SEQ ID NO. 7) | (SEQ ID NO. 8) |
| ACAN | GGCATCGTGTTCCATTACA | ACTCGTCCTTGTCTCCATAG |
| | G | (SEQ ID NO. 10) |
| | (SEQ ID NO. 9) | |
| SOSTDC1 | GTTCAAGTAGGCTGCCGAG | GCACTGGCCGTCTGAGATG |
| | AA | (SEQ ID NO. 12) |
| | (SEQ ID NO. 11) | |
| GAPDH | TGCCGCCTGGAGAAACC | CGCCTGCTTCACCACCTT |
| | (SEQ ID NO. 13) | (SEQ ID NO. 14) |

### Results

### Cell Viability

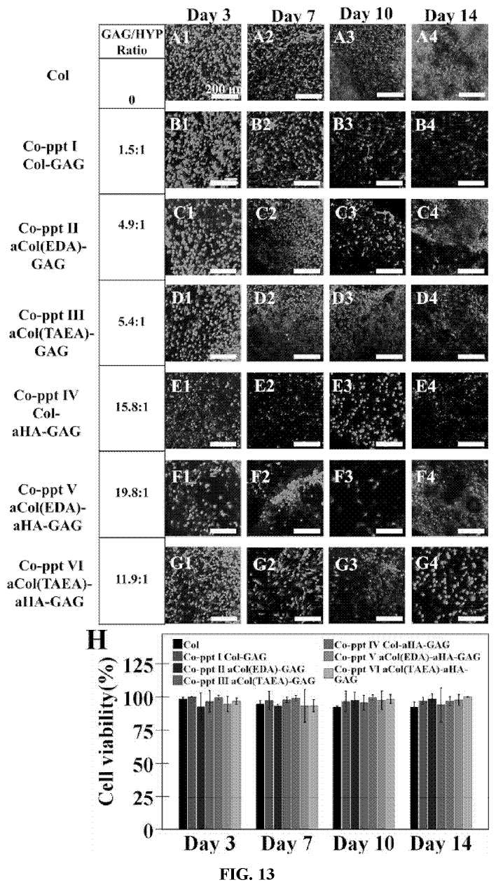

FIG. 13 shows the live/dead images of different coprecipitates. All the groups showed 93-100% cell viability was detected in aCol(EDA)-GAG and aCol(EDA)-aHA-GAG groups, suggesting the coprecipitates are biocompatible, supporting bNPCs survival and proliferation.

### Histological

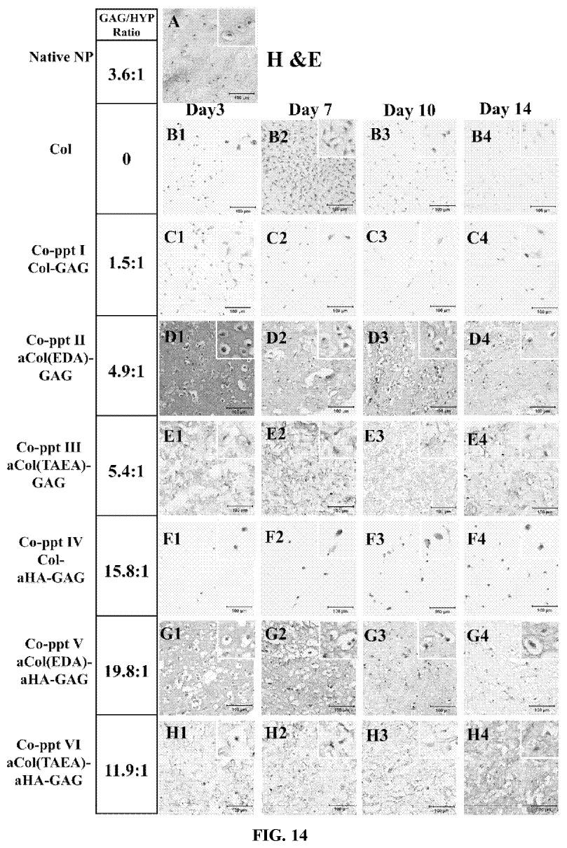

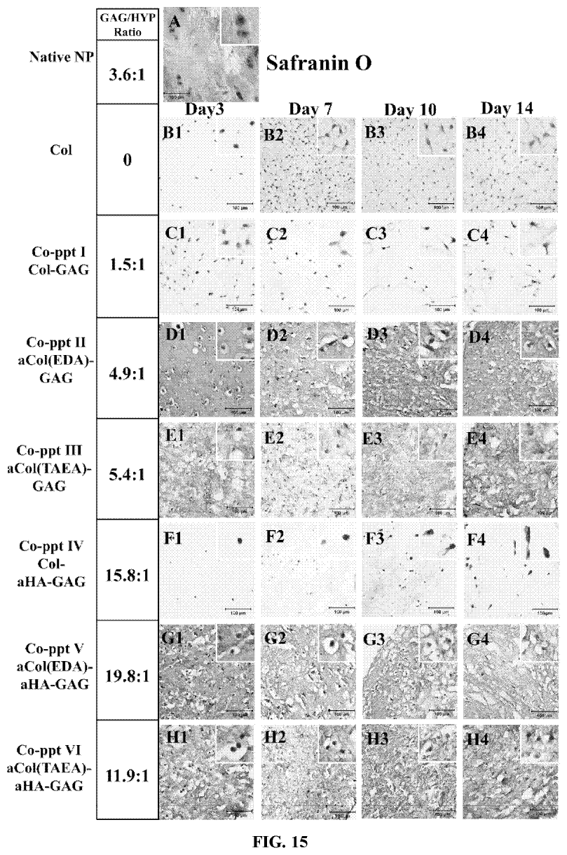

The H&E staining revealed the tissue anatomy, cell density, morphology, and distribution in which the bNPCs resided at Day 3, 7, 10, and 14 as well as native tissue. In native NP, round bNPCs are interspersed individually at a low concentration. bNPCs in collagen-GAG and collagen groups were elongated over time while the cells in aHA-collagen-GAG were still round. In aCol(EDA)-GAG, aCol(TAEA)-GAG, aCol(EDA)-aHA-GAG, aCol(TAEA)-aHA-GAG groups, cells were located in lacunae-like structures or form clusters in the collagen matrix (FIG. 14). As shown in FIG. 15, NP tissue was rich in the GAG. In collagen-GAG and aHA-collagen-GAG groups, the matrix was stained blue at all timepoints, which indicated that the matrix has rarely no GAG detected. In aCol(EDA)-GAG, aCol(TAEA)-GAG, aCol(EDA)-aHA-GAG, aCol(TAEA)-aHA-GAG groups, the matrix was strongly stained with Safranin-O at all time points and tended to increase overtime. The collagen microsphere showed negative staining at the beginning of day 3. Over time, cell density increased, and GAG deposited by cells was detected after 7 days of culture. These results suggested that aCol(EDA)-GAG, aCol(TAEA)-GAG, aCol(EDA)-aHA-GAG, aCol(TAEA)-aHA-GAG coprecipitates can structurally and functionally mimic the native NP.

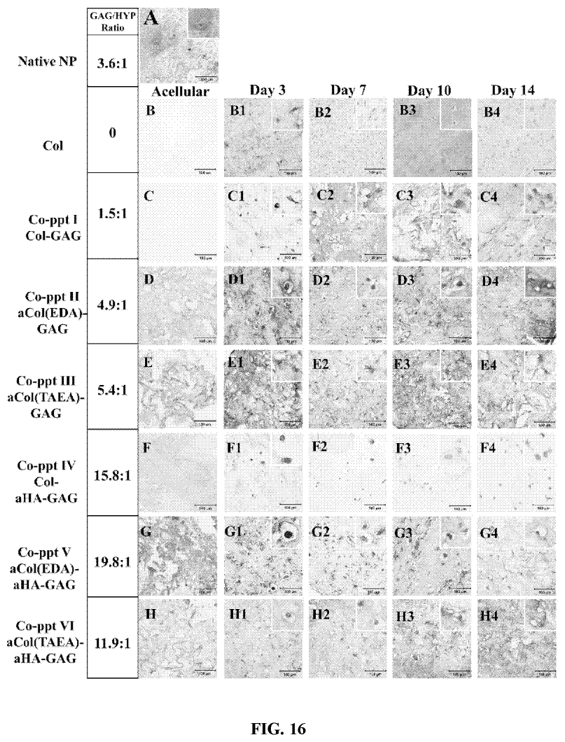

FIG. 16 shows the IHC staining of HA in the matrix of co-ppts. The native NP was abundant in HA that was staining with intensive HA staining. As expected, in acellular scaffolds, Col-aHA-GAG, aCol(EDA)-aHA-GAG, and aCol(TAEA)-aHA-GAG groups showed positive HA staining while the Col and Col-GAG groups showed negative HA staining. The aCol(EDA)-GAG and aCol(TAEA)-GAG groups were also positive staining with HA because of the similarity of GAG and HA. Upon bNPCs encapsulation, all the scaffolds were positively stained with HA secret by bNPCs, which can explain the TEM that the ‘bottlebrush’ structure appears in bNPCs encapsulated the aCol(EDA)-GAG and aCol(TAEA)-GAG groups.

### Cellular Phenotype Maintenance

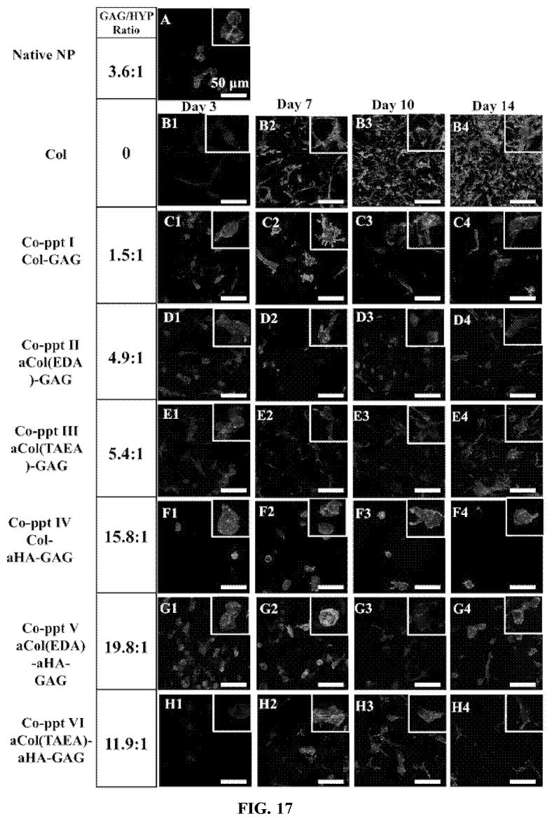

In native NP, actin was distributed as a weak ring around the periphery of cells (FIG. 17). In all groups, actin was observed around the periphery on day 3, similar to that in native tissue.

This actin pattern continued up to day 7 in aCol(EDA)-GAG, aCol(TAEA)-GAG, aCol(EDA)-aHA-GAG, aCol(TAEA)-aHA-GAG groups while the actin became longer and appeared as patches in Col and Col-GAG groups. On days 10 and 14, cells were elongated, and some stress fibers were observed in Col and Col-GAG groups but no obvious stress fibers in aCol(EDA)-GAG, aCol(TAEA)-GAG, aCol(EDA)-aHA-GAG, aCol(TAEA)-aHA-GAG groups. In Col-aHA-GAG groups showed the same round actin expression at all time points as native NP, suggesting that the actin maintains its pattern in the scaffold.

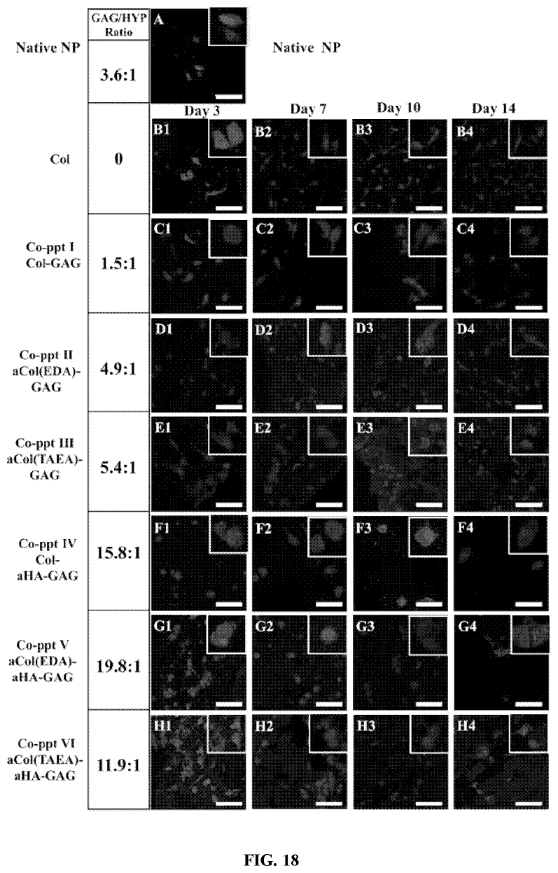

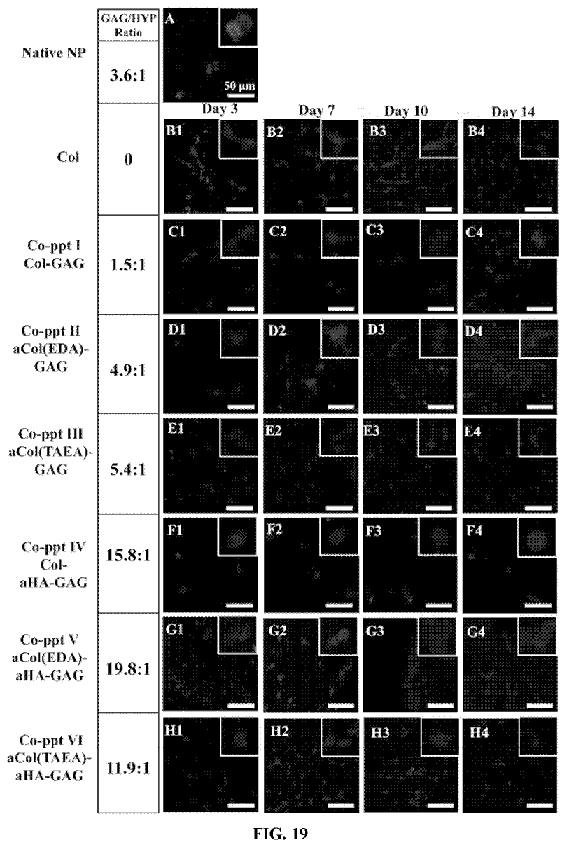

SNAP25 and KRT8 were reported as two potential phenotype markers of bNPCs and showed highly positive signals in native NP tissue (FIGS. 18 and 19). The expression of SNAP 25 and KRT8 was decreased from day 3 to day 14. The Col-aHA-GAG, aCol(EDA)-aHA-GAG and aCol(TAEA)-aHA-GAG coprecipitates showed extensively higher signal both SNAP 25 and KRT8 than Col-GAG, aCol(EDA)-GAG and aCol(TAEA)-GAG coprecipitates, indicated that the cellular phenotype was maintained in these scaffolds with aHA incorporation.

RT-qPCR

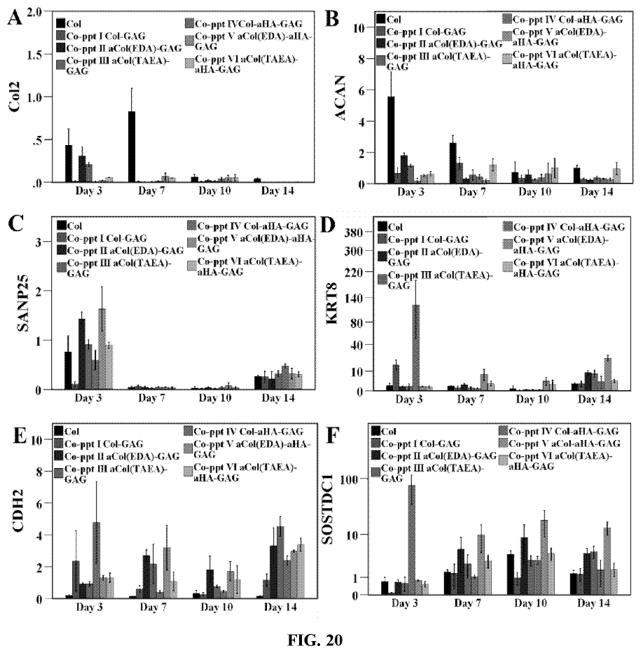

To study the phenotype maintenance of bNPCs in Co-ppts, the chondrogenic phenotypic markers Col2 and ACAN, and a series of non-chondrogenic phenotypic markers including SNAP25, KRT8, CDH2, and SOSTDC1 were investigated (FIG. 20). Col and ACAN were the matrix markers. Col and ACAN were downregulated during culture from day 3 to day 14, and the Col2 and ACAN expression of Col group were significantly higher than all the GAG incorporated Co-ppts, suggesting bNPCs might downregulate the matrix production once encapsulated into the Co-ppts upon GAG encapsulation.

The SNAP25, KRT8, CDH2, and SOSTDC1 were specific NPCs phenotype markers. The SNAP25 expression was downregulated from day 3 to day 10 but increased from day 10 to day 14. The expression of SNAP25 in aCol(EDA)-aHA-GAG was significantly higher than in other groups. The KRT8 expression of Col-GAG and Col-aHA-GAG was 15.3 and 120-fold at day 3 and rapidly downregulated to 0.8 and 0.6 at day 7. From day 7 to day 14, KRT 8 of aCol(EDA)-aHA-GAG was significantly higher than other groups. The same trend was found in CDH2 expression, Col-GAG and Col-aHA-GAG were 2.4 and 4.7-fold at day 3 and decreased to 0.6 and 0.4 on day 7. The CDH2 expression of aCol(EDA)-GAG and aCol(TAEA)-GAG was significantly higher than Col-GAG, and aCol(EDA)-aHA-GAG was significantly higher than Col-aHA-GAG from day 7 to day 10. The SOSTDC1 expression of Col-aHA-GAG was 75.3 at day and 1.0 at day 7. The SOSTDC1 expression of aCol(EDA)-aHA-GAG was significantly higher than other groups from day 7 to day 14 (p<0.001). These results suggested that the Co-ppts with aCol and aHA can maintain the bNPCs phenotype markers (SNAP25, KRT8, CDH2, and SOSTDC1).

### Example 7 Elastic Modulus Measurement of Acellular and Cell Encapsulated GAG-Rich Composition

### Materials and Methods

All coprecipitates, Col-GAG, aCol(EDA)-GAG, aCol(TAEA)-GAG, Col-aHA-GAG, aCol(EDA)-aHA-GAG, aCol(TAEA)-aHA-GAG were formed with 200 Kg of Col or aCol(EDA) or aCol(TAEA), 400 Kg of aHA, and 400 Kg of chondroitin-6-sulfate GAGS with or without bNPCs encapsulation were cut into 2 mm cylinder by 2 mm punch, native AF and native NP used as control. The elastic modulus was measured by the microplate compression method (Chan, Li et al. 2008).

### Results

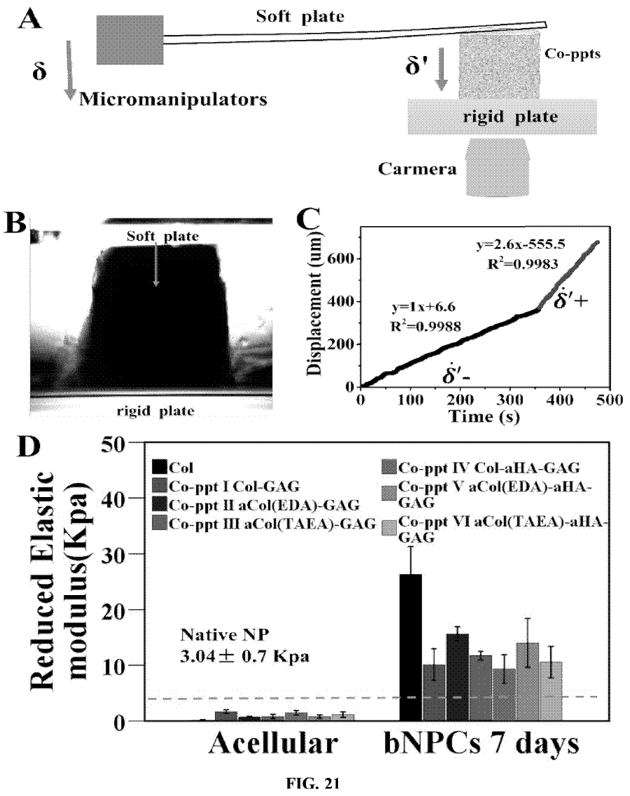

The reduced elastic modulus of the native tissue and GAG-rich scaffold was detected as an indicator for its physicochemical structural changes. As shown in FIG. 21, in native IVD, the elastic modulus of native AF was 16.31 Kpa, and that of native NP was 3.21 Kpa. In acellular scaffolds, the elastic modulus was all lower than native NP, which was 0.074 Kpa (Col), 1.64Kpa

(Col-GAG), 0.70 Kpa (aCol(EDA)-GAG), 0.81 Kpa (aCol(TAEA)-GAG), 1.47 Kpa (Col-aHA-GAG), 0.78Kpa (aCol(EDA)-aHA-GAG), 1.12 Kpa (aCol(TAEA)-aHA-GAG). After bNPCs encapsulated, the stiffness of scaffolds was increased to 26.33 Kpa(Col), 10.85 Kpa(Col-GAG), 15.65 Kpa (aCol(EDA)-GAG), 11.73 Kpa (aCol(TAEA)-GAG), 9.33 Kpa (Col-aHA-GAG), 12.38 Kpa (aCol(EDA)-aHA-GAG), 10.54 Kpa (aCol(TAEA)-aHA-GAG). The elastic modulus of bNPCs-encapsulated GAG scaffolds was lower than native AF and higher than native NP. These results indicated that the stiffness of the GAG-rich scaffolds was comparable to the native IVD, which can mechanically mimic the native tissue.

### Example 8 Osteogenic Differentiation of hMSCs in Scaffolds with Different GAG Contents

### Materials and Methods

Human MSCs (P2) purchased from ReachBio LLC (DBA: ReachBio Research Labs, USA) were cultured in Dulbecco's modified Eagle's medium (DMEM) (Gibco) containing low glucose and supplemented with 10% FBS (Gibco), 100 U/ml P/S (Gibco), and 2 mM 1-glutamine (Gibco) at 37° C. in a humidified incubator with 5% CO2, with regularly changed every 3-4 days. Cells at passage 5 were used for subsequent microencapsulation and investigation.

hMSCs were microencapsulated into scaffolds which were varied in GAG/HYP ratio: (I) Collagen (Col, GAG/HYP ratio 0); (II)Col-GAG (GAG/HYP ratio 1.5:1); (III) aCol(EDA)-GAG, named aCol-GAG (GAG/HYP ratio 4.9:1); (IV) aCol(EDA)-aHA-GAG, named aCol-aHA-GAG (with GAG/HYP ratio 6.8:1). Microencapsulation of hMSCs into Col microspheres was prepared as previously described(Hui, Cheung et al. 2008, Li, Choy et al. 2015). Briefly, hMSCs at a cell density of 5E5 cells/ml were mixed with NaOH neutralized type I Col solution (BD Biosciences, Bedford, MA, USA) to a final concentration of 2 mg/ml. Droplets of 4 μl of the mixture were pipetted to a Petri dish (Sterilin Ltd., Newport, UK) and incubated at 37° C. For scaffold II, the GAG at a final concentration of 1 mg/ml (the initial weight ratio of Col/GAG was 1:2) was mixed with hMSCs (5E5/m1) and Col (2 mg/ml), and 4 μl hMSCs-Col-GAG microspheres were formed. For scaffolds I and II, 50 microspheres were aggregated into an F-127 (Sigma—Aldrich) coated U-shape 96-well plate for the subsequent differentiation. Scaffold III and IV have used another microencapsulation method, called co-precipitation. To microencapsulation of hMSCs into aCol-GAG and aCol-aHA-GAG, 2.5E4 of hMSCs were mixed with 100 μg of aCol, 200 μg of GAGs, and 400 μg of aHA (scaffold IV).

The hMSCs microencapsulated in the 4 scaffolds were induced to undergo osteogenic differentiation and cultured in an induction medium consisting of DMEM low glucose basal medium that supplemented with 10% FBS (Gibco), 100 U/ml P/S (Gibco), 100 nM dexamethasone (Sigma-Aldrich), 10 ng/ml Bone morphogenetic protein-2 (BMP2, PeproTech, Inc.), 10 mM Beta-glycerophosphate (Sigma-Aldrich Co. LLC), and 50 nM Ascorbic Acid (Fluka, St. Louis, MO, USA) (Cheng, Luk et al. 2011). On time point days 7, 14, and 21, samples were harvested for characterization.

On days 7, 14, and 21, hMSCs encapsulated in Col, Col-GAG, aCol-GAG, and aCol-aHA-GAG were fixed with 4% PFA for 30 mins and then cut into 10 μm paraffin sections. The H&E staining was then used to reveal the cell morphology, and the Von Kossa staining was used to reveal the calcium-GAG region. For the Von Kossa staining, the sections were briefly incubated with 1% silver nitrate solution (Sigma) and were irradiated under ultraviolet light for 1 h. Unreacted silver was removed by incubating with 2% sodium thiosulfate for 5 mins. Nuclear fast red was used as a counterstain.

On day 21, samples were rinsed with PBS thrice and fixed with 4% PFA(Sigma-Aldrich) 4° C. for overnight. On the one hand, the fixed samples were dehydrated with gradient ethanol (10%, 30%, 50%, 70%, 90%, 95%, and 100%, 30 mins each) and thoroughly dried by critical point drying, followed by sputter-coating gold, and imaged using SEM (S-4800, Hitachi, Tokyo). On the other hand, the fixed samples were then processed to embedding into the epoxy resin and cut ultra-thin sections in 100 nm thickness. And then, the ultra-thin sections were stained with 2% aqueous uranyl acetate and Reynold's lead citrate. The ultrastructure of cells and the precellular matrix were examined with transmitting electron microscopy (TEM, Philips CM100).

The expression levels of major osteogenic markers, including ALP, BMP2, and RUNX2, were investigated to determine whether the hMSCs differentiation into the osteogenic lineage. Table 2 shows the sequence of the primers used for evaluation. On days 7, 14, and 21, hMSCs-encapsulated constructs were harvested, and the RT-qPCR was performed to measure the gene expression of the ALP, BMP2, and RUNX2.

TABLE 2
The primers used in the RT-PCR.

| Gene name | Forward primer | Reverse primer |
|---|---|---|
| hGAPDH | GAGTCAACGGATTTGGT | TTGATTTTGGAGGGATCTCG |
| | CGT | (SEQ ID NO. 16) |
| | (SEQ ID NO. 15) | |

Osteogenic differentiation

| | | |
|---|---|---|
| hRUNX2 | ACAGTAGATGGACCTCG | TGAGGCGGTCAGAGAACAAA |
| | GGAAC | (SEQ ID NO. 18) |
| | (SEQ ID NO. 17) | |
| hALP | CGCACGGAACTCCTGAC | GCCACCACCACCATCTCG |
| | C | (SEQ ID NO. 20) |
| | (SEQ ID NO. 19) | |
| hBMP2 | GAGGTCCTGAGCGAGTT | TCTCTGTTTCAGGCCGAACA |
| | CGA | (SEQ ID NO. 22) |
| | (SEQ ID NO. 21) | |

### Results

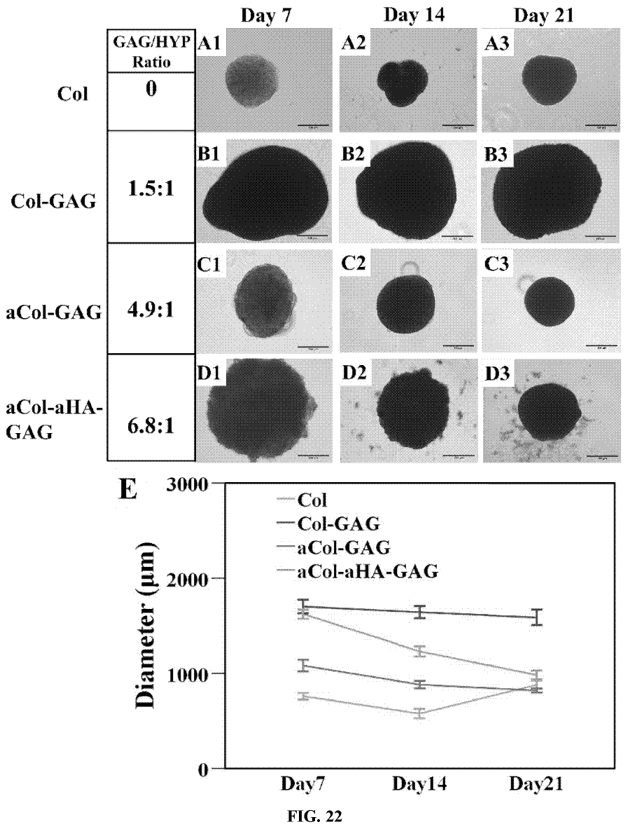

Morphologies Change hMSCs were microencapsulated into the Col, Col-GAG, aCol-GAG, and aCol-aHA-GAG, and the morphologies of these hMSCs-scaffolds were different from each other. FIG. 22 showed the gross appearance of hMSCs encapsulated scaffolds and osteogenic differentiated for different times. In general, the diameter of the Col microspheres was reduced from day 7 to day 14, and it increased as the Col microspheres started to form aggregates from day 14 to day 21. The diameter of the Col-GAG, aCol-GAG and aCol-aHA-GAG groups was reduced over time during differentiation.

### Histological and Histochemical Analysis

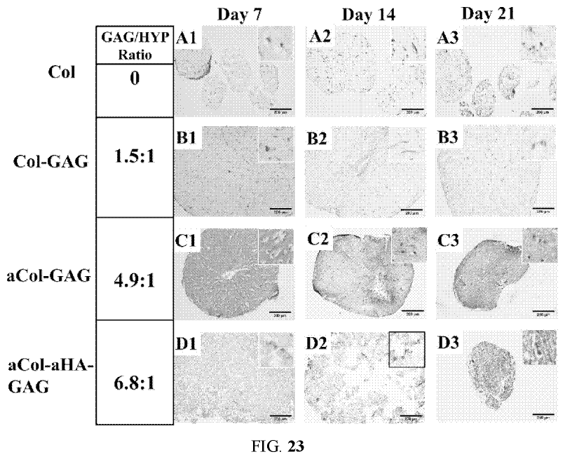

FIG. 23 showed the H&E staining of the hMSCs encapsulated scaffolds upon osteogenic differentiation. In general, microspheres upon osteogenic differentiation for 14 days and 21 days showed hypocellularity with the basophilic matrix in comparison with 7 days. By contrast, the matrix of the aCol-GAG and aCol-aHA-GAG groups was more condensed than that of the Col and Col-GAG groups.

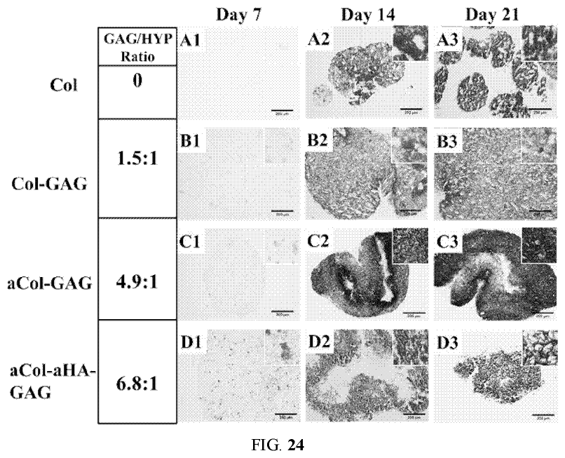

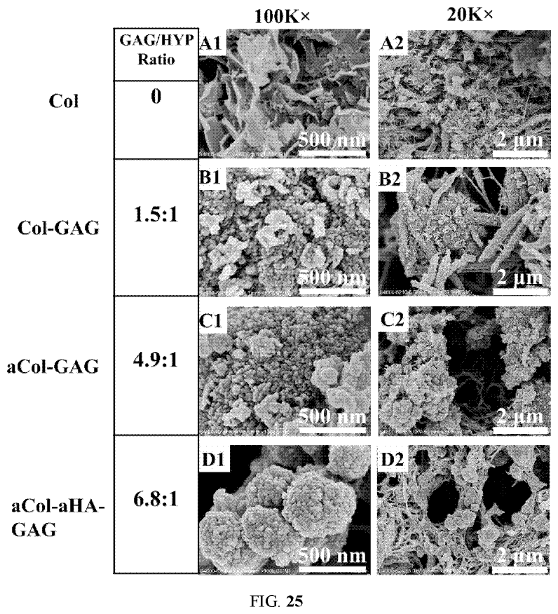

FIG. 24 showed the Von Kossa staining of osteogenic differentiated scaffolds, revealing detectable mineral deposition. Brown to black color was observed in all groups, suggesting a large amount of calcium was deposited in all groups from day 14 to day 21. By comparison, the intensity of Von Kossa staining of the Col group was higher than that of the Col-GAG group, and the aCol-GAG group was higher than that of the aCol-aHA-GAG group, suggesting higher calcium deposition in Col and aCol-GAG groups.

### Ultrastructural Analysis

SEM and EDX were conducted to measure the ultrastructure of the hMSCs-encapsulated scaffolds upon osteogenic differentiation. FIG. 25 showed the SEM images of hMSCs encapsulated into Col, Col-GAG, aCol-GAG, and aCol-aHA-GAG scaffolds. Upon osteogenic differentiation, numerous calcium granules are deposited within the collagen fiber meshwork. The morphology was varied in Col, Col-GAG, aCol-GAG, and aCol-aHA-GAG groups. Specifically, calcium deposition with the ‘nano-flower’-like structures was found in the Col group. In the Col-GAG group, the calcium was ‘rod’-like structures at the low magnification (20K×), and that was the ‘nanobeads’ structures and aggregations of ‘nanobeads’ structures under high magnification (100K×). The aCol-GAG and aCol-aHA-GAG groups were abundant in ‘nanobeads’ aggregates structures. These results suggested that incorporating GAGS altered the ultrastructure of calcium as the highly negatively charged GAGS may interact with these minerals.

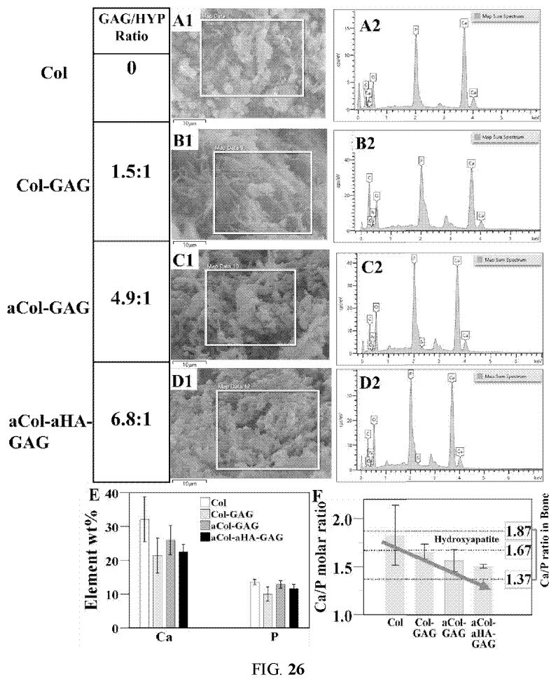

FIG. 26 showed the EDX mapping and quantification analysis of calcium and phosphorus elements in Col, Col-GAG, aCol-GAG, and aCol-aHA-GAG scaffolds. The Col group showed a higher weight percentage in both Ca and P elements (32.1±6.6%, and 13.6±0.8%, respectively) than those of the Col-GAG (21.4±5.2%, and 10±0.8%, respectively) group. The weight percentage of Ca and P elements was 25.9±3.7% and 12.8+1.0% in the aCol-GAG group, 22.5±2.3% and 11.6±1.3% in the aCol-aHA-GAG group. FIG. 26F showed the calculated Ca/P molar ratio in the Col, Col-GAG, aCol-GAG, and aCol-aHA-GAG groups. The Ca/P molar ratio of the Col, Col-GAG, aCol-GAG, and aCol-aHA-GAG groups was 1.82±0.31, 1.66±0.08, 1.56±0.10, 1.50±0.19, respectively. The Ca/P ratio of the mineral in healthy bone was reported at a range of 1.37 to 1.87 (Hing 2004), and the prominent component of the inorganic matrix in bone, hydroxyapatite Cal 0(PO4)6(OH)2, has a Ca/P ratio of 1.67 (Pellegrino and Biltz 1968). Only the Col group showed a higher Ca/P ratio than the hydroxyapatite (1.67).

Real-Time qPCR Analysis

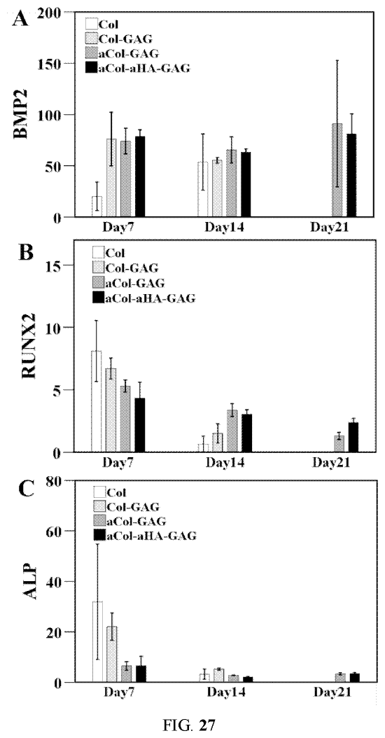

The gene expression of the osteogenic differentiation phenotype markers including BMP2, RUNX2, and ALP was shown in FIG. 27. BMP2 is one of the osteoinductive growth factors that participate in the regulation of cell differentiation. The BMP2 expression was not affected by the variety of time and culture time. As the soluble BMP2 was used during osteogenic differentiation, the BMP2 expression was at a stable level.

The RUNX2 expression was downregulated over time. Two-way ANOVA showed the RUNX2 expression was significantly different during culture time (p<0.001). Specifically, on day 7, the RUNX2 expression of the Col (8.1-fold) group was significantly higher than the aCol-GAG (5.3-fold) and aCol-aHA-GAG (4.3-fold) groups, suggesting the Col group higher osteogenic differentiation ability in the early stage of osteogenic differentiation.

Alkaline phosphatase (ALP) is one of the earliest markers for osteoblasts that regulate the mineralization of the matrix. In general, the ALP level was downregulated during osteogenic differentiation from day 7 to day 21. The ALP expression of Col was significantly higher than the aCol-GAG and aCol-aHA-GAG groups rather than the Col-GAG group.

### Example 9 Chondrogenic Differentiation of hMSCs in Compositions with Different GAG Contents

### Materials and Methods

Human MSCs at passage 4 were used for chondrogenic differentiation and subsequent microencapsulation and investigation. hMSCs were microencapsulated into scaffolds which were varied in GAG/HYP ratio: (I) Collagen (Col, GAG/HYP ratio 0); (II)Col-GAG (GAG/HYP ratio 1.5:1); (III) aCol(EDA)-GAG, named aCol-GAG (GAG/HYP ratio 4.9:1); (W) aCol(EDA)-aHA-GAG, named aCol-aHA-GAG (with GAG/HYP ratio 6.8:1). And the Col concentration used was 2 mg/ml. Hence, in the aCol-GAG and aCol-aHA-GAG groups, 1E5 of hMSCs were mixed with 100 μg of aCol, 200 μg of GAGs, and 400 μg of aHA (scaffold W).

For chondrogenic differentiation, the induction medium consisted of DMEM high glucose basal medium that supplemented with 100 U/ml P/S (Gibco), 10 ng/ml recombinant human transforming growth factor-β3 (TGF-β3, Merck, Darmstadt, Germany), 1.25 mg/ml BSA (Sigma-Aldrich Co. LLC), ITS-A premix (Merck & Co), 1 mM sodium pyruvate (Gibco), and 0.35 mM L-proline (Merck & Co. Inc.). On days 7, 14, and 28, samples were harvested for histological staining and ultrastructural analysis as described in Example 7.

TABLE 3
The primers used in the RT-PCR.

| Gene name | Forward primer | Reverse primer |
|---|---|---|
| hGAPDH | GAGTCAACGGATTTGG | TTGATTTTGGAGGGATCTCG |
| | TCGT | (SEQ ID NO. 16) |
| | (SEQ ID NO. 15) | |

Chondrogenic differentiation

| | | |
|---|---|---|
| SOX9 | CGCCATCTTCAAGGCG | CCTGGGATTGCCCCGAGTGC |
| | CTGC | (SEQ ID NO. 24) |
| | (SEQ ID NO. 23) | |
| Col2 | GGCAATAGCAGGTTCA | CGATAACAGTCTTGCCCCAC |
| | CGTACA | TT |
| | (SEQ ID NO. 25) | (SEQ ID NO. 26) |
| ACAN | ACAGCTGGGGACATTA | GTGGAATGCAGAGGTAATTT |
| | GTGG | (SEQ ID NO. 28) |
| | (SEQ ID NO. 27) | |

The expression levels of major chondrogenic markers, including COL2, ACAN, and SOX9, were investigated to determine whether the hMSCs differentiation into the chondrogenic lineage. Table 3 shows the sequence of the primers used for evaluation. On days 7, 14, and 28, hMSCs-encapsulated constructs were harvested, and the RT-qPCR was performed.

### Results

### Morphologies Change

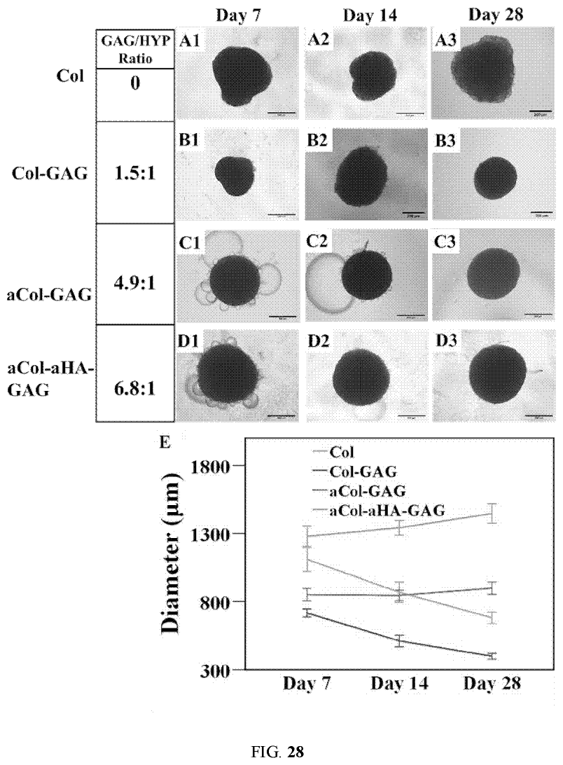

As shown in FIG. 28, the spherical morphology of microspheres was maintained over time. In general, the diameter of the Col and Col-GAG groups was significantly decreased due to the contraction of hMSCs over time. On the other hand, the diameter of the aCol-GAG and aCol-aHA-GAG groups was increased from day 7 to day 28. Specifically, there were numerous small ‘bubbles’ surrounding the microspheres in the aCol-GAG and aCol-aHA-GAG groups from day 7 to day 14, mainly due to the GAGs-water interaction.

### Histological and Histochemical Analysis

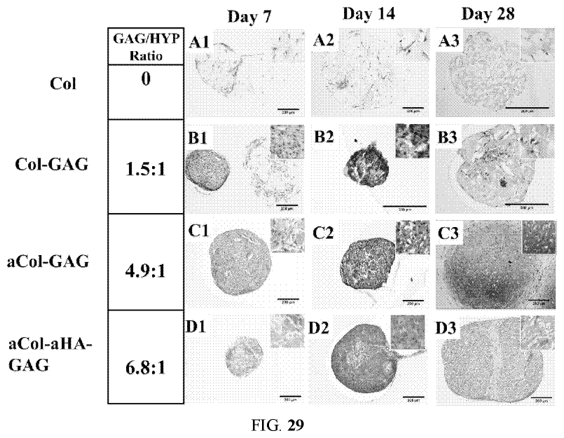

As shown in FIG. 29, H&E staining revealed the typical cellular and matrix morphology of the cartilage-like tissues. In the Col group, cells were elongated, with a small amount of matrix deposition. Cell clusters were found in the Col-GAG group over time. Specifically, in high GAG content scaffolds, the aCol-GAG, and aCol-aHA-GAG groups, cells were round and located in lacuna-like structures, similar to that of the native cartilage. In comparison, the aCol-GAG group demonstrated better chondrocytes-like morphology than other groups.

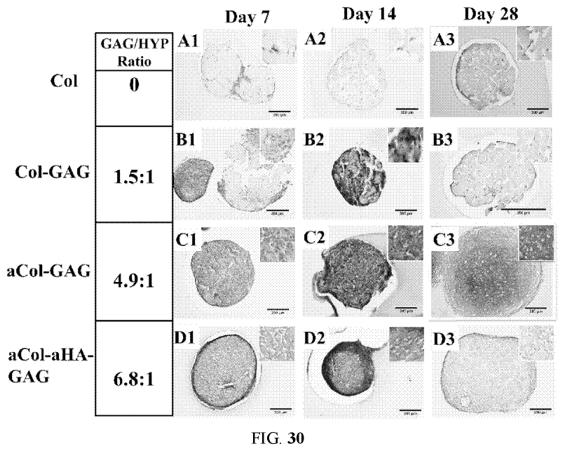

The ECM of cartilage is abundant in GAGS, and the Safranin 0 staining was conducted to reveal the GAG-rich cartilage-like regions (FIG. 30). The abundance of GAG in the 4 scaffolds was different. The Col and Col-GAG groups showed negative GAG staining with green color during differentiation from day 7 to day 28, while the aCol-GAG and aCol-aHA-GAG groups showed highly positive staining of GAGS with red color. In the short-term (day 7), the positive GAG staining of aCol-GAG and aCol-aHA-GAG was due to the scaffold formation that the GAG/HYP ratio of initial aCol-GAG and aCol-aHA-GAG groups was 4.9:1 and 6.8:1, respectively, in the long-term (day 14-28) upon chondrogenic differentiation, the GAG secretion and deposition increased, particularly, in the aCol-GAG group on day 28, the GAG intensity was significantly higher than other groups.

### Ultrastructural Analysis

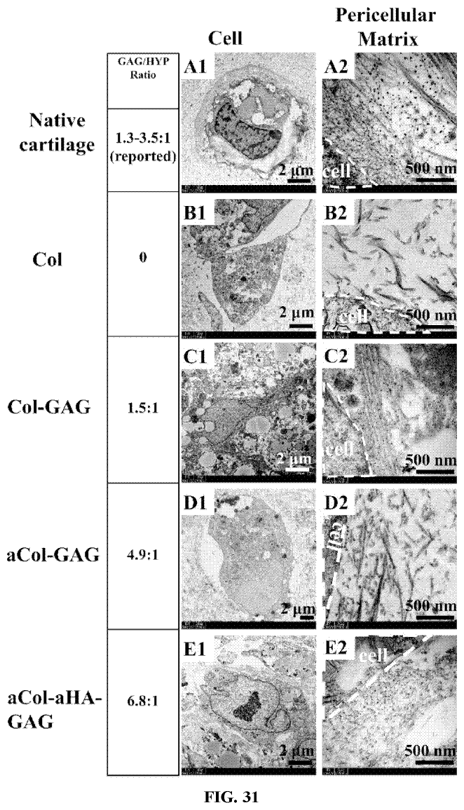

FIG. 31 showed the TEM images that reveal the ultrastructure of cells, the fibrous meshwork, and the pericellular matrix of the native cartilage and hMSCs encapsulated scaffolds with chondrogenic differentiation for 28 days. In native cartilage, chondrocytes were round and resided in a lacuna structure, and a lamella structure was around the chondrocytes, called the pericellular matrix. As shown in FIG. 31A2, abundance ‘nanobeads’ bound to fibrous meshwork was found in the pericellular matrix of chondrocytes, representing GAGS and Col fibers, respectively.

Upon 28 days of chondrogenic differentiation, the morphology of cells and pericellular matrix were varied in 4 scaffolds. In the Col group, the cells were elongated, and the truncated collagen fibrils without ‘nanobeads’ were found around cells, suggesting no GAGS deposition in the pericellular matrix. The cells were also elongated in the Col-GAG group, but the fibrous meshwork was much like that of the native cartilage, with numerous ‘nanobeads’ attached to the collagen fibers. On the other hand, cells were more round in the aCol-GAG and aCol-aHA-GAG groups, abundant ‘nanobeads’ along the Col fiber was found in the pericellular matrix of these two groups, suggesting these two scaffolds have high GAG incorporation were structurally mimicking the native cartilage, and the incorporation of GAG was a benefit for chondrogenic differentiation.

Real-Time qPCR Analysis

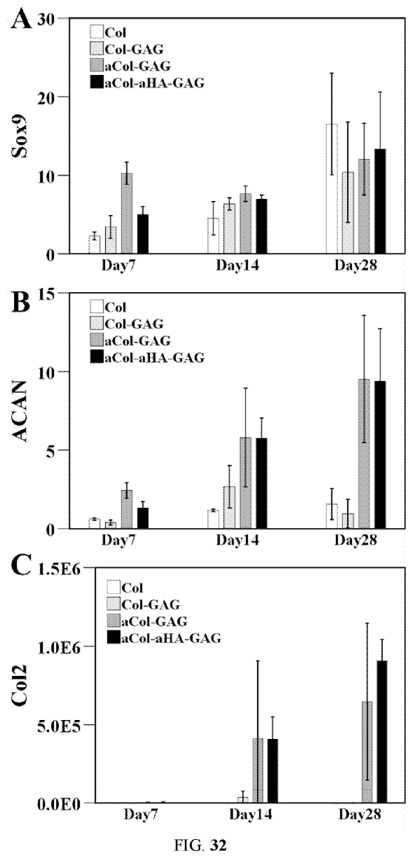

The phenotypic changes of hMSCs in 4 scaffolds upon chondrogenic differentiation were revealed by the expression level of the chondrogenic markers, including SOX9, ACAN, and Col2 (FIG. 32). SOX9 is a transcription factor that plays a crucial role in chondrogenesis. In general, the SOX9 expression was upregulated during chondrogenic differentiation from day 7 to day 28. Specifically, on day 7, the SOX9 expression of the aCol-GAG group was significantly higher than that of other groups, suggesting that cells in the aCol-GAG group differentiated earlier than other groups.

The ACAN expression of hMSCs chondrogenic differentiated into 4 scaffolds were shown in FIG. 32B. Also, the ACAN expression was upregulated over time. The ACAN expression of the aCol-GAG and aCol-aHA-GAG groups was significantly higher than that of the Col and Col-GAG. The same trend was found in the Col2 expression that it was also upregulated over time (FIG. 32C).

The Col2 expression of the aCol-GAG and aCol-aHA-GAG groups was significantly higher than that of the Col and Col-GAG groups. These results suggested that the incorporation of GAGS promotes chondrogenic differentiation.

### Example 10 Discogenic Differentiation of hMSCs in Compositions with Different GAG Contents

### Materials and Methods

Human MSCs at passage 4 were used for discogenic differentiation and subsequent microencapsulation and investigation. hMSCs were microencapsulated into scaffolds which were varied in GAG/HYP ratio: (I) Collagen (Col, GAG/HYP ratio 0); (II)Col-GAG (GAG/HYP ratio 1.5:1); (III) aCol(EDA)-GAG, named aCol-GAG (GAG/HYP ratio 4.9:1); (W) aCol(EDA)-aHA-GAG, named aCol-aHA-GAG (with GAG/HYP ratio 19.8:1).

For discogenic differentiation, the induction medium consisted of DMEM high glucose basal medium that supplemented with 100 U/ml P/S (Gibco), 10 ng/ml recombinant Growth Differentiation Factor 5 (GDFS, PeproTech, Inc.), 1.25 mg/ml BSA (Sigma-Aldrich Co. LLC), ITS-A premix (Merck & Co), 1 mM sodium pyruvate (Gibco), and 0.35 mM L-proline (Merck & Co. Inc.). On days 7, 14, and 28, samples were harvested for histological staining and ultrastructural analysis as described in Example 7.

The expression levels of major chondrogenic markers, including COL2, ACAN, PAX1, and FOXF1 were investigated to determine whether the hMSCs differentiation into the NP-like lineage. Table 4 shows the sequence of the primers used for evaluation. On days 7, 14, and 28, hMSCs-encapsulated constructs were harvested, and the RT-qPCR was performed.

TABLE 4
The primers used in the RT-PCR.

| Gene name | Forward primer | Reverse primer |
|---|---|---|
| hGAPDH | GAGTCAACGGATTTGG | TTGATTTTGGAGGGATCTCG |
| | TCGT | (SEQ ID NO. 16) |
| | (SEQ ID NO. 15) | |

Discogenic differentiation

| | | |
|---|---|---|
| Col2 | GGCAATAGCAGGTTCA | CGATAACAGTCTTGCCCCAC |
| | CGTACA | TT |
| | (SEQ ID NO. 25) | (SEQ ID NO. 26) |
| ACAN | ACAGCTGGGGACATTA | GTGGAATGCAGAGGTAATTT |
| | GTGG | (SEQ ID NO. 28) |
| | (SEQ ID NO. 27) | |
| hPAX1 | AAGCCGCCCTATTCCT | GCGCTTGGTGGGTGAACT |
| | ACATC | (SEQ ID NO. 30) |
| | (SEQ ID NO. 29) | |
| hFOXF1 | AAGCCGCCCTATTCCT | GCGCTTGGTGGGTGAACT |
| | ACATC | (SEQ ID NO. 32) |
| | (SEQ ID NO. 31) | |

### Results

### Morphologies Change

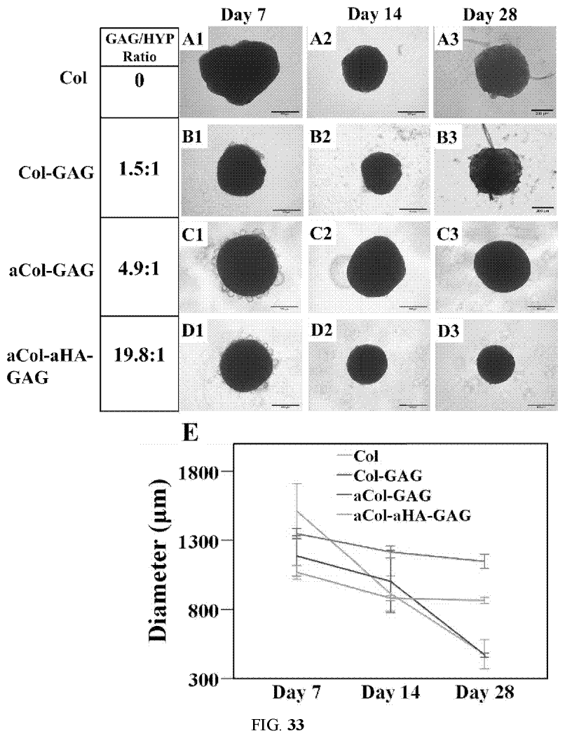

FIG. 33 showed the gross appearance of the scaffolds upon discogenic differentiation. By comparison, the Col and Col-GAG groups showed a similar trend to the chondrogenic ones that the diameter of which was significantly decreased over time. The diameter of the aCol-GAG and aCol-aHA-GAG groups showed a slight reduction over time, but the reduction was significantly less than that of the Col and Col-GAG groups. The small ‘bubbles’ were also observed in the aCol-GAG and aCol-aHA-GAG scaffolds on day 7 and day 14, and the GAG-water interaction helped these two scaffolds maintain their volume.

### Histological and Histochemical Analysis

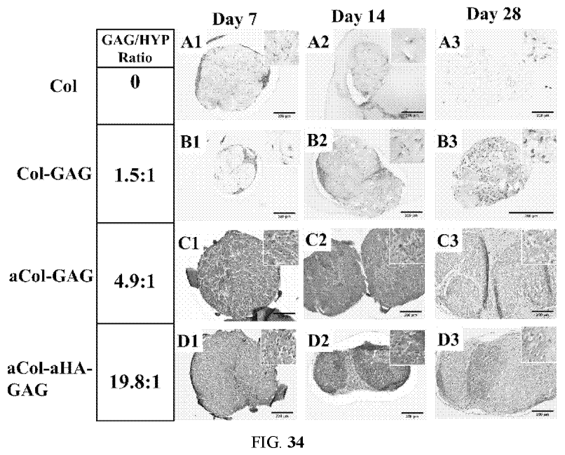

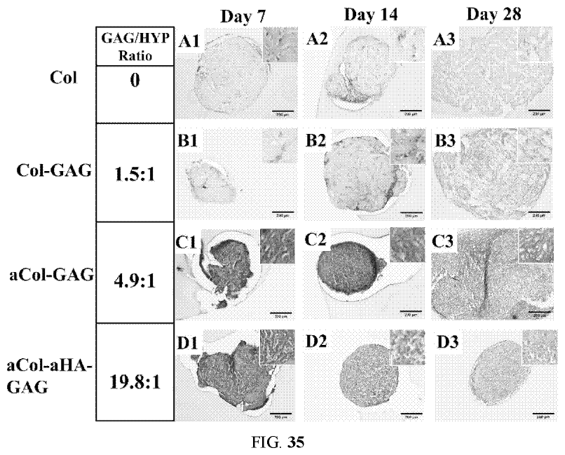

The H&E staining (FIG. 34) of hMSCs encapsulated scaffolds and discogenic differentiated for 7, 14, and 28 days showed some trend as the chondrogenic one. The cell density and matrix deposition of the aCol-GAG and aCol-aHA-GAG groups was higher than the Col and Col-GAG groups. Cells in the Col group were more elongated, while cells in the Col-GAG group showed clusters, both with a small amount of matrix deposition over time. By comparison, cells in the aCol-GAG and aCol-aHA-GAG groups were round, with a large amount of matrix deposited.

The abundance of GAG of hMSCs encapsulated scaffolds was investigated by Safranin 0 staining (FIG. 35). In general, the GAG-positive region and intensity in the aCol-GAG and aCol-aHA-GAG groups were higher than that of the Col and Col-GAG groups. The Col group showed negative GAG staining with the blue color over time from day 7 to day 28. Although the Col-GAG group started to show positive GAG staining on day 28, the intensity was far below the aCol-GAG and aCol-aHA-GAG groups. The aCol-GAG and aCol-aHA-GAG groups showed intensive positive staining with red color over time, suggesting high GAG incorporation and deposition.

### Ultrastructural Analysis

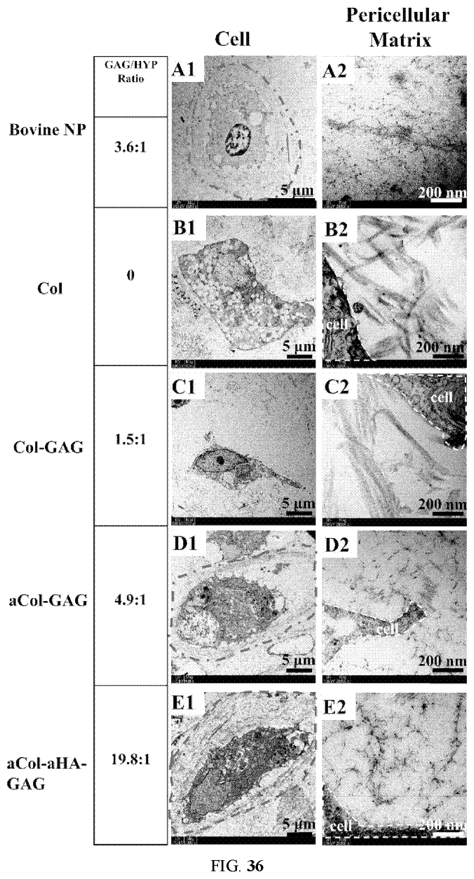

FIG. 36 showed TEM images of the native bovine NP and hMSCs encapsulated scaffolds under discogenic differentiation for 28 days. The GAG/HYP ratio of the native NP (27:1) was significantly higher than that of the native cartilage (3.1-4.2:1), and hence the ultrastructure of the NP was different from that of the cartilage. Herein, bovine NP was used as an NP example. In native bovine NP, the NP cells were round with an obviously circular pericellular matrix. The native NP was observed as the unregular small ‘bottlebrush’ structures, suggesting that the pericellular matrix of native NP was more abundant in PGs and GAGS, but not Col. In the Col group, the cells were hypertrophic, and no apparent pericellular matrix circle was observed that the matrix surrounding cells were composed of Col fibers. In the Col-GAG group, the cells were elongated, and the Col fibers with typical D-bands were observed surrounding the cells. In the aCol-GAG and aCol-aHA-GAG groups, cells were round and surrounded by pericellular matrix lamella, and the ‘bottlebrush’ structures without Col fibers was overserved in the pericellular matrix that was similar to the native NP.

Real-Time qPCR Analysis

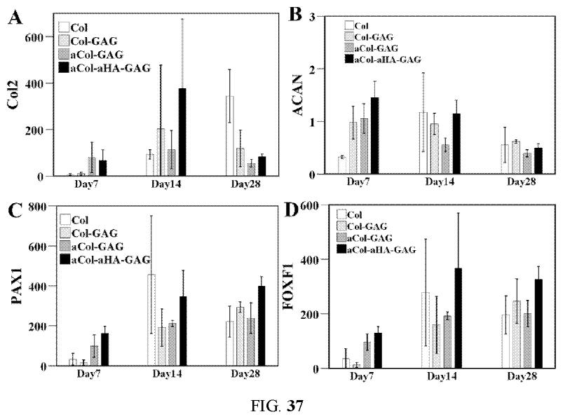

FIG. 37 showed the gene expression of discogenic differentiation markers, including the chondrogenic markers, Col2 and ACAN, and the NPC-specific markers, PAX1, and FOXF1. FIGS. 37A and B showed the Col2 and ACAN expression in 4 scaffolds. In general, the expression of Col2 was upregulated from day 7 to day 14 and then downregulated from day 14 to day 28, while the ACAN was downregulated over time. Specifically, on day 7, the aCol-GAG and aCol-aHA-GAG groups showed significantly higher expression of Col2 than the Col and Col-GAG groups. In addition, the aCol-GAG and aCol-aHA-GAG groups showed significantly higher expression of ACAN than that of the Col group. From day 14 to day 28, no significant differences were found among groups. These results suggested that the aCol-aHA-GAG and aCol-GAG groups with high GAG incorporation were upregulated the expression of the chondrogenic markers, such as Col2 and ACAN, in the early differentiation.

Paired box 1 (PAX1) is a transcription factor that regulates pattern formation during embryogenesis invertebrates, whereas the Forkhead box F1 (FOXF1) plays an essential role in cell growth, proliferation, and differentiation. FIGS. 37C and D showed the expression of PAX1 and FOXF1; generally, the expression of PAX1 and FOXF1 was upregulated over time. On day 7, the aCol-GAG and aCol-aHA-GAG groups showed significantly higher expression of PAX1 and FOXF1 than the Col and Col-GAG groups. Specifically, on day 28, the aCol-aHA-GAG group showed significantly higher PAX1 and FOXF1 than the Col and aCol-GAG (groups. These results demonstrated that the aCol-aHA-GAG (GAG/HYP 19.8:1) promotes the expression of the discogenic markers.

### Example 11 In Vivo Biocompatibility of the GAG-Rich Composition

### Materials and Methods

All procedures of the animal study were conducted in the animal unit and approved by the Animal Research Ethics Committee of the University of Hong Kong. Female nude mouse (6-weeks old) with bodyweight of 18-25 g was used. After shaving, an incision was made at the back, and a subcutaneous pocket was created. Acellular samples: (I) Col; (II) Col-GAG; (III) aCol-GAG; (IV) Col-aHA-GAG; and (V) aCol-aHA-GAG, and hMSCs-encapsulated and discogenic differentiation for 28 days samples: (I) Col; (II) Col-GAG; (III) aCol-GAG; and (W) aCol-aHA-GAG was implanted separately. The wound was then closed immediately with an absorbable suture. The scaffolds were retrieved at 1-month post-implantation, fixed in 4% PFA, and sectioned for subsequent H&E and Safranin 0 staining.

### Results

### Acellular Scaffolds

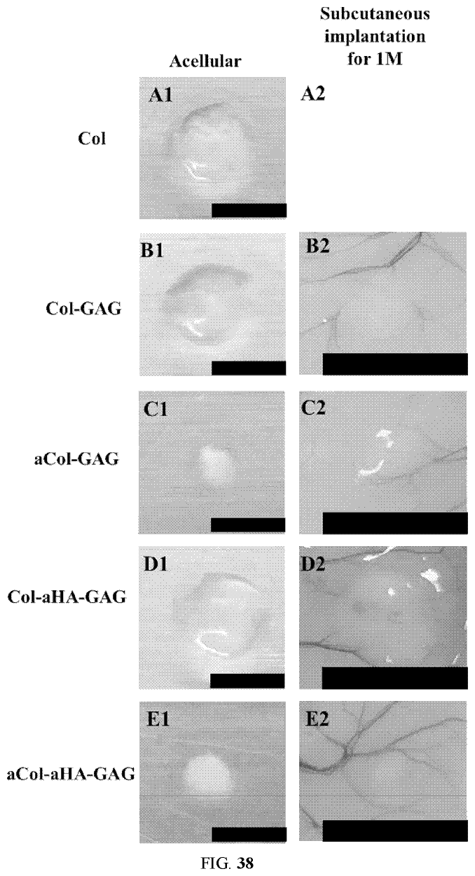

The GAG-rich scaffolds showed good biocompatibility up to 1 month after subcutaneous implantation in nude mice (FIG. 38). Before implantation, the gross appearance of the scaffolds showed gel-like structures. After implantation for 1 month, the Col scaffold disappeared as the Col was easily absorbed in vivo. On the other hand, the GAG-contained scaffolds were all intact in a gel-like structure.

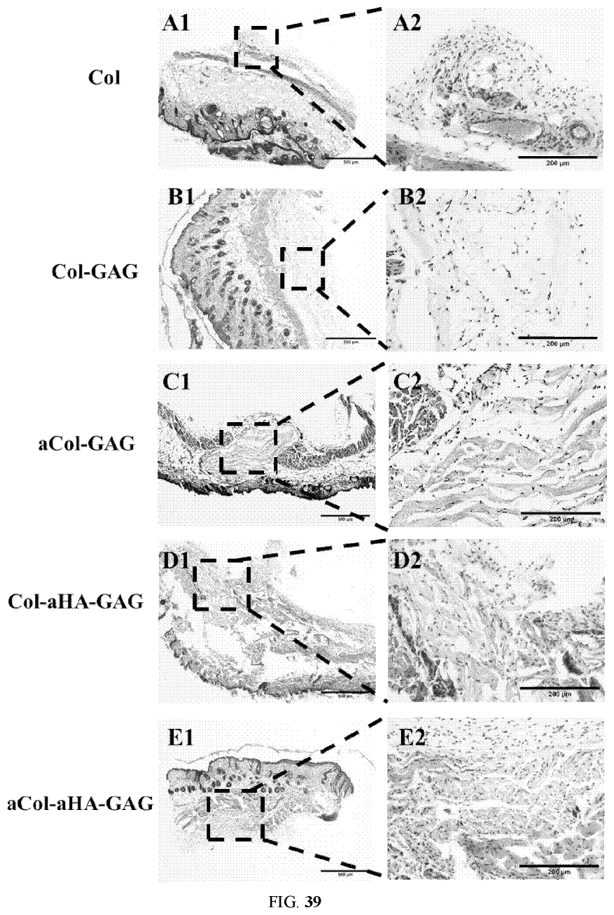

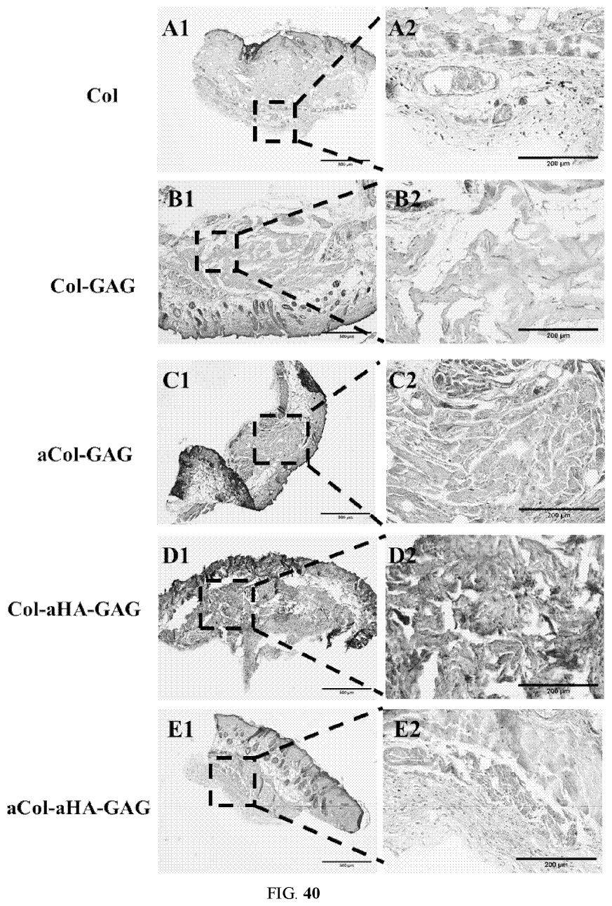

Histologically, the GAG contained scaffolds were all showed good biocompatibility that elongated fibroblastic cells were found that integrated with the scaffolds (FIG. 39). All the GAG-contained scaffolds had no signs of inflammation or foreign body reaction. With safranin 0 staining, the GAG positive region was observed (FIG. 40). In the Col-GAG and Col-aHA-GAG groups, the scaffolds were GAG-negative stained in green color. On the other hand, in the aCol-GAG and aCol-aHA-GAG groups, the scaffolds were GAG-positive in red Color. These results suggested that the aCol-GAG and aCol-aHA-GAG can integrate with the native cells and tissue and maintain the high GAG content for at least one month, enabling its application as cell-free scaffolds for GAG-rich tissue regeneration.

hMSCs-Scaffolds

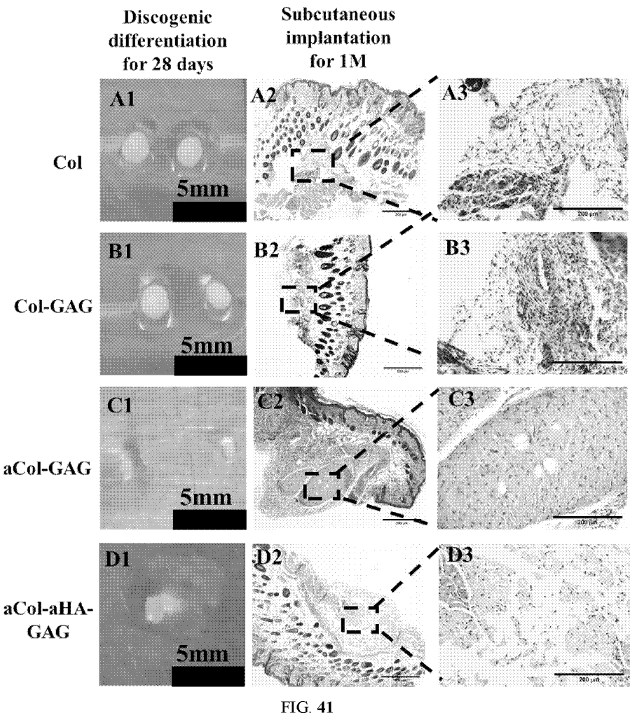

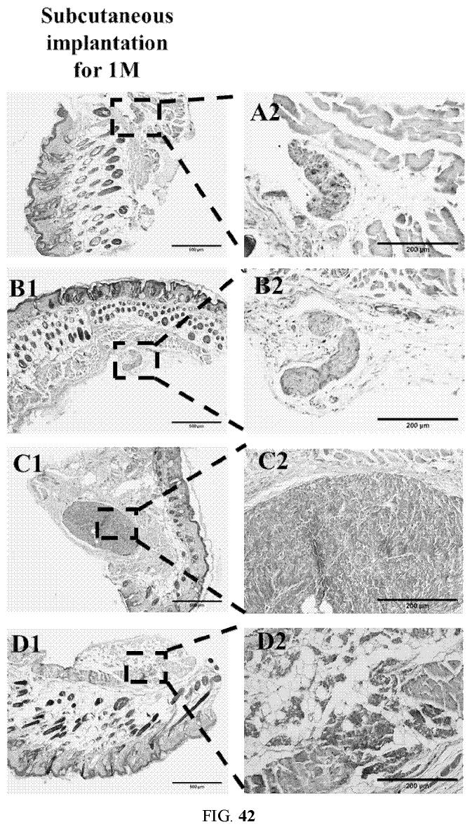

The hMSCs-encapsulated scaffolds also showed good biocompatibility (FIG. 38). Before implantation, the gross appearance of the hMSCs-encapsulated scaffolds was in a spherical structure. After implantation for 1 month, all the hMSCs-encapsulated scaffolds reduced their volume and integrated with the skin of the nude mouse. All the 4 scaffolds showed no signs of inflammation or foreign body reaction (FIG. 41). The abundance of GAG of hMSCs-encapsulated scaffolds after 1-month implantation was investigated by Safranin 0 staining (FIG. 42). In general, the GAG-positive region and intensity in the aCol-GAG and aCol-aHA-GAG groups were higher than that of the Col and Col-GAG groups, suggesting these two groups had higher GAG content. These results indicated that the aCol-GAG and aCol-aHA-GAG scaffold the great potential in used as the cell carrier for GAG-rich tissue engineering.

### REFERENCES

Barbosa, I., S. Garcia, V. Barbier-Chassefiere, J. P. Caruelle, I. Martelly and D. Papy-Garcia (2003). “Improved and simple micro assay for sulfated glycosaminoglycans quantification in biological extracts and its use in skin and muscle tissue studies.” Glycobiology 13(9): 647-653.
Brougham, C. M., T. J. Levingstone, S. Jockenhoevel, T. C. Flanagan and F. J. O'Brien (2015). “Incorporation of fibrin into a collagen-glycosaminoglycan matrix results in a scaffold with improved mechanical properties and enhanced capacity to resist cell-mediated contraction.” Acta Biomater 26: 205-214.
Caliari, S. R. and B. A. Harley (2014). “Collagen-GAG scaffold biophysical properties bias MSC lineage choice in the presence of mixed soluble signals.” Tissue Eng Part A 20(17-18): 2463-2472.
Chan, B. P., T. Y. Hui, C. W. Yeung, J. Li, I. Mo and G. C. Chan (2007). “Self-assembled collagen-human mesenchymal stem cell microspheres for regenerative medicine.” Biomaterials 28(31): 4652-4666.
Chan, W. C., K. L. Sze, D. Samartzis, V. Y. Leung and D. Chan (2011). “Structure and biology of the intervertebral disk in health and disease.” Orthop Clin North Am 42(4): 447-464, vii.
Cheng, H. W., K. D. K. Luk, K. M. C. Cheung and B. P. Chan (2011). “In vitro generation of an osteochondral interface from mesenchymal stem cell-collagen microspheres.” Biomaterials 32(6): 1526-1535.
Choy, A. T., K. W. Leong and B. P. Chan (2013). “Chemical modification of collagen improves glycosaminoglycan retention of their co-precipitates.” Acta Biomater 9(1): 4661-4672.
Grier, W. K., E. M. Iyoha and B. A. C. Harley (2017). “The influence of pore size and stiffness on tenocyte bioactivity and transcriptomic stability in collagen-GAG scaffolds.” J Mech Behav Biomed Mater 65: 295-305.
Hing, K. (2004). “Bone repair in the twenty-first century: Biology, chemistry or engineering?”

Philosophical transactions. Series A, Mathematical, physical, and engineering sciences 362: 2821-2850.
Homicz, M. R., K. B. McGowan, L. M. Lottman, G. Beh, R. L. Sah and D. Watson (2003). “A compositional analysis of human nasal septal cartilage.” Arch Facial Plast Surg 5(1): 53-58.
Hui, T. Y., K. M. Cheung, W. L. Cheung, D. Chan and B. P. Chan (2008). “In vitro chondrogenic differentiation of human mesenchymal stem cells in collagen microspheres: influence of cell seeding density and collagen concentration.” Biomaterials 29(22): 3201-3212.
Jana, P., T. Mitra, T. K. R. Selvaraj, A. Gnanamani and P. P. Kundu (2016). “Preparation of guar gum scaffold film grafted with ethylenediamine and fish scale collagen, cross-linked with ceftazidime for wound healing application.” Carbohydr Polym 153: 573-581.
Kirk, J. F., G. Ritter, I. Finger, D. Sankar, J. D. Reddy, J. D. Talton, C. Nataraj, S. Narisawa, J. L. Millan and R. R. Cobb (2013). “Mechanical and biocompatible characterization of a cross-linked collagen-hyaluronic acid wound dressing.” Biomatter 3(4).
Li, Y. Y., T. H. Choy, F. C. Ho and P. B. Chan (2015). “Scaffold composition affects cytoskeleton organization, cell-matrix interaction and the cellular fate of human mesenchymal stem cells upon chondrogenic differentiation.” Biomaterials 52: 208-220.
Liu, M., Y. Xu, C. Huang, T. Jia, X. Zhang, D. P. Yang and N. Jia (2018). “Hyaluronic acid-grafted three-dimensional MVVCNT array as biosensing interface for chronocoulometric detection and fluorometric imaging of CD44-overexpressing cancer cells.” Mikrochim Acta 185(7): 338.
Mullen, L. M., S. M. Best, S. Ghose, J. Wardale, N. Rushton and R. E. Cameron (2015). “Bioactive IGF-1 release from collagen-GAG scaffold to enhance cartilage repair in vitro.” J Mater Sci Mater Med 26(1): 5325.
Mwale, F., P. Roughley and J. Antoniou (2004). “Distinction between the extracellular matrix of the nucleus pulposus and hyaline cartilage: a requisite for tissue engineering of intervertebral disc.” Eur Cell Mater 8: 58-63; discussion 63-54.
Pellegrino, E. D. and R. M. Biltz (1968). “Bone carbonate and the Ca to P molar ratio.” Nature 219(5160): 1261-1262.
Silagi, E. S., I. M. Shapiro and M. V. Risbud (2018). “Glycosaminoglycan synthesis in the nucleus pulposus: Dysregulation and the pathogenesis of disc degeneration.” Matrix Biol 71-72: 368-379.
Tian, Y., W. Yuan, J. Li, H. Wang, M. G. Hunt, C. Liu, I. M. Shapiro and M. V. Risbud (2016). “TGFbeta regulates Galectin-3 expression through canonical Smad3 signaling pathway in nucleus pulposus cells: implications in intervertebral disc degeneration.” Matrix Biol 50: 39-52.
Treppo, S., H. Koepp, E. C. Quan, A. A. Cole, K. E. Kuettner and A. J. Grodzinsky (2000). “Comparison of biomechanical and biochemical properties of cartilage from human knee and ankle pairs.” J Orthop Res 18(5): 739-748.
van Weeren, P. R. and E. C. Firth (2008). “Future tools for early diagnosis and monitoring of musculoskeletal injury: biomarkers and CT.” Vet Clin North Am Equine Pract 24(1): 153-175.
Woessner, J. F., Jr. (1961). “The determination of hydroxyproline in tissue and protein samples containing small proportions of this imino acid.” Arch Biochem Biophys 93: 440-447.
Zhong, S., W. E. Teo, X. Zhu, R. Beuerman, S. Ramakrishna and L. Y. Yung (2005). “Formation of collagen-glycosaminoglycan blended nanofibrous scaffolds and their biological properties.” Biomacromolecules 6(6): 2998-3004.
Zhou, Y., B. Yang, X. Ren, Z. Liu, Z. Deng, L. Chen, Y. Deng, L. M. Zhang and L. Yang (2012). “Hyperbranched cationic amylopectin derivatives for gene delivery.” Biomaterials 33(18): 4731-4740.

### Patents

Dan Simionescu, Jeremy J. Mercuri(inventor). Clemson University(assignee). Shape-memory sponge hydrogel biomaterial. U.S. Pat NO. U.S. Pat. No. 9,283,301B1. Mar. 15, 2016.
Ioannis V. Yannas, Philip L. Gordon Chor Huang, Frederick H. Silver, John F. Burke(inventor). Massachusetts Institute of Technology(assignee). Crosslinked collagen-mucopolysaccharide composite materials. U.S. Pat NO. U.S. Pat. No. 4,280,954A. Jul. 28, 1981.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

1. A composition comprising a glycosaminoglycan component, and one or more extracellular matrix components forming a precipitate with the glycosaminoglycan component, wherein the precipitate has a glycosaminoglycan to hydroxyproline ratio from about 1:10 to about 100:1.

2. The composition according to claim 1, wherein the one or more extracellular matrix components are selected from the group consisting of collagen, hyaluronic acid, fibronectin, laminin, a core protein, a link protein, a peptide, a derivative thereof, a salt thereof, and a combination thereof.

3. The composition according to claim 1, wherein the one or more extracellular matrix components comprise collagen, hyaluronic acid, a derivative thereof, and/or a salt thereof, and at least one extracellular matrix component has a functional group reacting with the glycosaminoglycan component for forming the precipitate.

4. The composition according to claim 1, wherein the glycosaminoglycan component is selected from sulfated glycosaminoglycan, heparin/heparan sulfate, chondroitin sulfate, dermatan sulfate, keratan sulfate, a derivative thereof, or a combination thereof.

5. The composition according to claim 1, wherein the one or more extracellular matrix components have one or more amino groups for reacting with the glycosaminoglycan component to form the precipitate, and the one or more extracellular matrix components are positively charged or neutral.

6. The composition according to claim 1, wherein the extracellular matrix components comprise aminated collagen, and aminated hyaluronic acid.

7. The composition according to claim 6, wherein the collagen is aminated by an amination reagent selected from ethylenediamine (EDA), tris(2-aminoethyl) amine (TAEA), L-arginine, metformin, or a polyamine, and preferably the amination reagent is ethylenediamine or tris(2-aminoethyl) amine.

8. The composition according to claim 1, wherein the precipitate has the glycosaminoglycan to hydroxyproline ratio of from 1:1 to 100:1, from 1:1 to 50:1, preferably 5:1 or 27:1.

9. The composition according to claim 1, wherein the precipitate shows GAG retention within 1 to 100 days for 1% to 99%, preferably 50%.

10. The composition according to claim 1, wherein the precipitate is in the form of small nano-sized ‘beads’ like structure, micro-scale aggregation, or ‘bottlebrush’ like structure.

11. The composition according to claim 1, wherein the precipitate has the glycosaminoglycan to hydroxyproline ratio of 5:1 for promoting chondrogenic differentiation of stem cells, or of 7:1 for promoting discogenic differentiation of stem cells.

12. A method of making a composition of claim 1, comprising the steps of:
(i) providing one or more extracellular matrix components, optionally the one or more extracellular matrix components are aminated by an amination reagent;
(ii) purifying the one or more extracellular matrix components optionally by dialysis;
(iii) providing an aqueous solution of one or more extracellular matrix components;
(iv) mixing the aqueous solution of step (iii) with a glycosaminoglycan component to form a precipitate, optionally followed by shaking or vortexing; and
(v) collecting the precipitate;
wherein the precipitate has a glycosaminoglycan to hydroxyproline ratio from about 1:10 to about 100:1.

13. The method of claim 12, wherein the amination reagent is a cationic chemical comprising at least two primary amino groups.

14. The method of claim 13, wherein the amination reagent is ethylenediamine (EDA), tris(2-aminoethyl) amine (TAEA), L-arginine, metformin, a polyamine, or a combination thereof.

15. A method of treating a tissue disorder in a subject comprising administering the composition according to claim 1 to the subject, wherein the composition serves as a swelling agent and/or a volume filing agent for implanting into a GAG-rich tissue in the subject.

16. The method of claim 15, wherein the GAG-rich tissue is nucleus pulposus (NP) or cartilage.

17. A method of culturing a tissue having abundant glycosaminoglycans, comprising a step of providing the composition of claim 1 as a substrate, a cell-free scaffold, or a cell-microcarrier.

18. The method of claim 17, wherein the tissue is nucleus pulposus (NP) or cartilage.

19. A device comprising the composition of claim 1.

20. The device of claim 19, further comprising stem cells, or cells isolated from cartilage, bones and nucleus pulposus.
